(19) United States
(12) Patent Application Publication
(10) Pub. No.: US 2012/0328565 A1
(43) Pub. Date: Dec. 27, 2012

(54) ANTIVIRAL COMPOUNDS

(76) Inventors: John A. Brinkman, West Caldwell, NJ (US); Andrew F. Donnell, West Windsor, NJ (US); Robert Francis Kester, West Orange, NJ (US); Yimin Qian, Wayne, NJ (US); Ramakanth Sarabu, Towaco, NJ (US); Sung-Sau So, Verona, NJ (US)

(21) Appl. No.: 13/528,920

(22) Filed: Jun. 21, 2012

Related U.S. Application Data
(60) Provisional application No. 61/500,640, filed on Jun. 24, 2011.

(51) Int. Cl. A61K 31/4178; C07D 417/14; C07D 401/14; C07D 487/04; C12N 5/071; A61K 31/4439; A61K 31/551; A61K 38/21; A61P 31/14; C07D 403/14; A61K 31/428

(52) U.S. Cl. 424/85.4; 548/314.7; 548/178; 546/272.7; 540/500; 514/397; 514/367; 514/341; 514/221; 435/375

(57) ABSTRACT

The present invention discloses compounds of Formula I

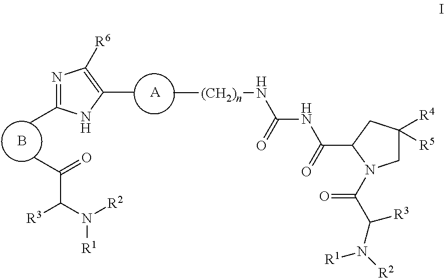

wherein the variables in Formula I are defined as described herein. Also disclosed are pharmaceutical compositions containing such compounds and methods for using the compounds of Formula I in the treatment of HCV infection.

### PRIORITY TO RELATED APPLICATIONS

This application is entitled to the benefit of U.S. provisional patent application Ser. No. 61/500,640 filed on Jun. 24, 2011.

### FIELD OF THE INVENTION

The present invention provides non-nucleoside compounds of Formula I useful as inhibitors of hepatitis C virus (HCV), as inhibitors of HCV replication, and for the treatment of hepatitis C infection.

Hepatitis C virus (HCV) infection is a major health problem that leads to chronic liver disease, such as cirrhosis and hepatocellular carcinoma, in a substantial number of infected individuals. Current treatments for HCV infection include immunotherapy with recombinant interferon-α alone or in combination with the nucleoside-analog ribavirin.

Several virally-encoded enzymes are putative targets for therapeutic intervention, including a metalloprotease (NS2-3), a serine protease (NS3, amino acid residues 1-180), a helicase (NS3, full length), an NS3 protease cofactor (NS4A), a membrane protein (NS4B), a zinc metalloprotein (NS5A) and an RNA-dependent RNA polymerase (NS5B).

One identified target for therapeutic intervention is HCV NS5A non-structural protein. A non-structural protein, NS5A is an essential component for viral replication and assembly. Mutations in NS5A at or near known sites of phosphorylation can affect the ability for high-level replication in cell-culture systems, suggesting an important role for NS5A phosphorylation in viral replication efficiency. Inhibitors of the phosphorylation of NS5A can lead to reduced viral RNA replication.

There is a clear and long-felt need to develop effective therapeutics for treatment of HCV infection. Specifically, there is a need to develop compounds that are useful for treating HCV-infected patients and compounds that selectively inhibit HCV viral replication.

### SUMMARY OF THE INVENTION

The application provides a compound of Formula I

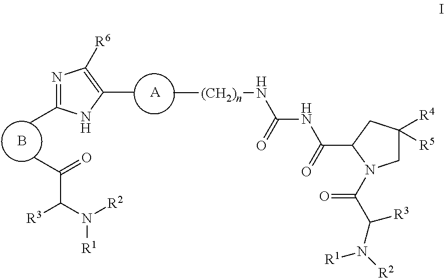

wherein:
R¹ and R² are independently H, lower alkyl, COOR⁷, COR^{S}, or CONHR⁹;
R³ is lower alkyl or aryl;
R⁴ and R⁵ are independently H or halo;
R⁶ is H or halo;
n is 0 or 1;
R⁷, R⁸, and R⁹ are independently H or lower alkyl;

### A is

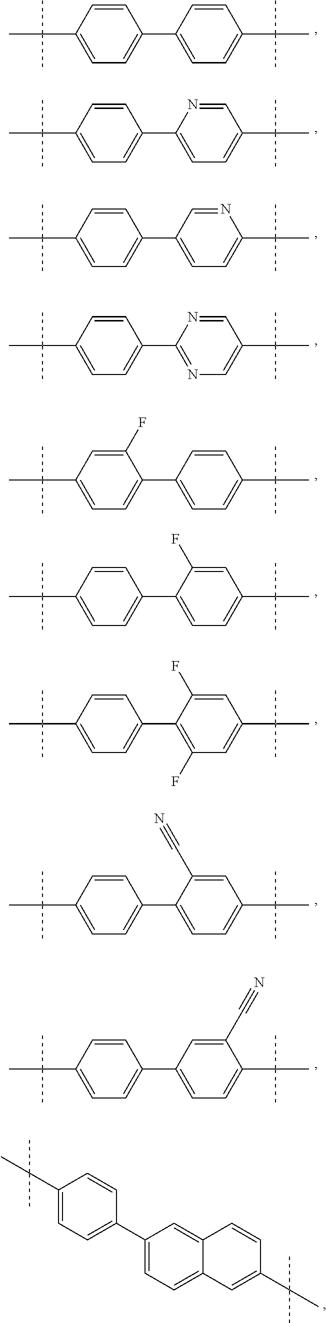

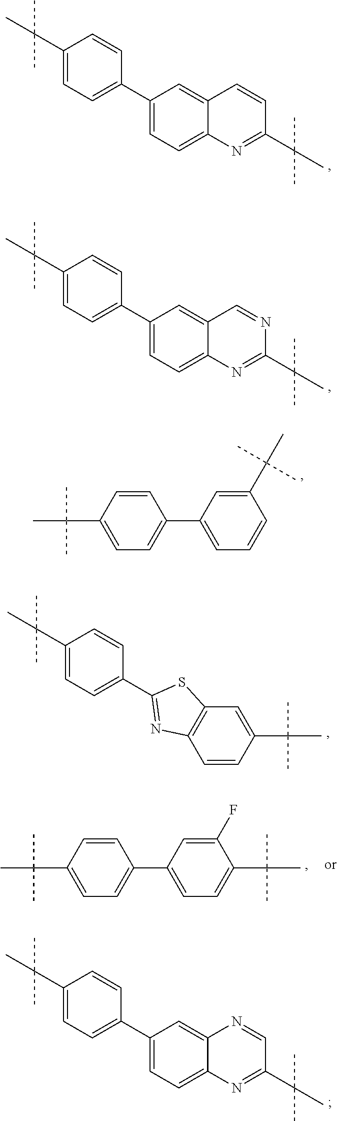

and

### B is

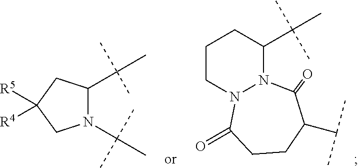

or a pharmaceutically acceptable salt thereof.

The application provides a method for treating a Hepatitis C Virus (HCV) infection comprising administering to a patient in need thereof a therapeutically effective amount of a compound of Formula I.

The application provides a composition comprising a compound of any one of Formula I and a pharmaceutically acceptable excipient.

### DETAILED DESCRIPTION OF THE INVENTION

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “- - - ” drawn through a bond each refer to the point of attachment of a functional group or other chemical moiety to the rest of the molecule of which it is a part. Thus, for example:
MeC(═O)OR⁴ wherein R⁴=

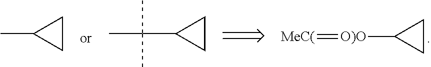

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen atom or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—√—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “spirocycloalkyl”, as used herein, means a spirocyclic cycloalkyl group, such as, for example, spiro[3.3]heptane. The term spiroheterocycloalkyl, as used herein, means a spirocyclic heterocycloalkyl, such as, for example, 2,6-diaza spiro[3.3]heptane.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “ester” as used herein denotes a group of formula —C(═O)OR wherein R is lower alkyl as defined herein.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″-, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an -O-alkyl wherein alkyl is C₁₋₁₀.

The term “PCy₃” refers to a phosphine trisubstituted with three cyclic moieties.

The terms “haloalkoxy” or “halo-lower alkoxy” or “lower haloalkoxy” refers to a lower alkoxy group, wherein one or more carbon atoms are substituted with one or more halogen atoms.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic or partially unsaturated ring containing four to eight atoms per ring, incorporating one or more N, O, or S heteroatoms, the remaining ring atoms being carbon, with the understanding that the attachment point of the heteroaryl radical will be on an aromatic or partially unsaturated ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, oxazinyl, pyrrolyl, pyrazolyl, imidazolyl, oxazolyl, 4,5-Dihydro-oxazolyl, 5,6-Dihydro-4H-[1,3]oxazolyl, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole, naphthyridinyl, 5,6,7,8-Tetrahydro-[1,6]naphthyridinyl, and benzisothiazole. Bicyclic moieties can be optionally substituted on either ring, however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, and ionic forms thereof, unless otherwise indicated. Examples of heterocyclic radicals include, but are not limited to, morpholinyl, piperazinyl, piperidinyl, azetidinyl, pyrrolidinyl, hexahydroazepinyl, oxetanyl, tetrahydrofuranyl, tetrahydrothiophenyl, oxazolidinyl, thiazolidinyl, isoxazolidinyl, tetrahydropyranyl, thiomorpholinyl, quinuclidinyl and imidazolinyl, and ionic forms thereof. Examples may also be bicyclic, such as, for example, 3,8-diaza-bicyclo[3.2.1]octane, 2,5-diaza-bicyclo[2.2.2]octane, or octahydro-pyrazino[2,1-c][1,4]oxazine.

### Inhibitors of HCV NSSA

The application provides a compound of Formula I

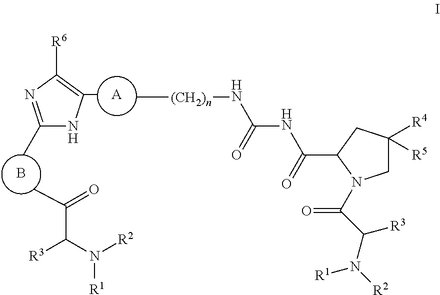

wherein:
R¹ and R² are independently H, lower alkyl, COOR⁷, COR^{S}, or CONHR⁹;
R³ is lower alkyl or aryl;
R⁴ and R⁵ are independently H or halo;
R⁶ is H or halo;
n is 0 or 1;
R⁷, R⁸, and R⁹ are independently H or lower alkyl;

### A is

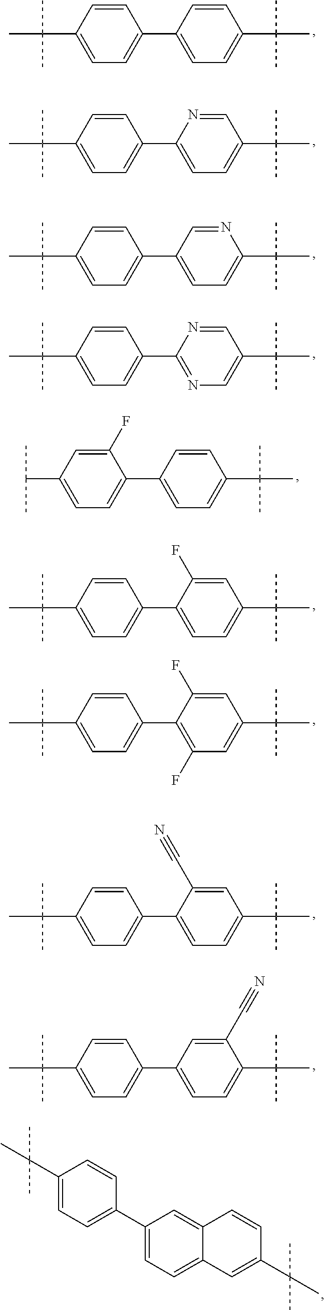

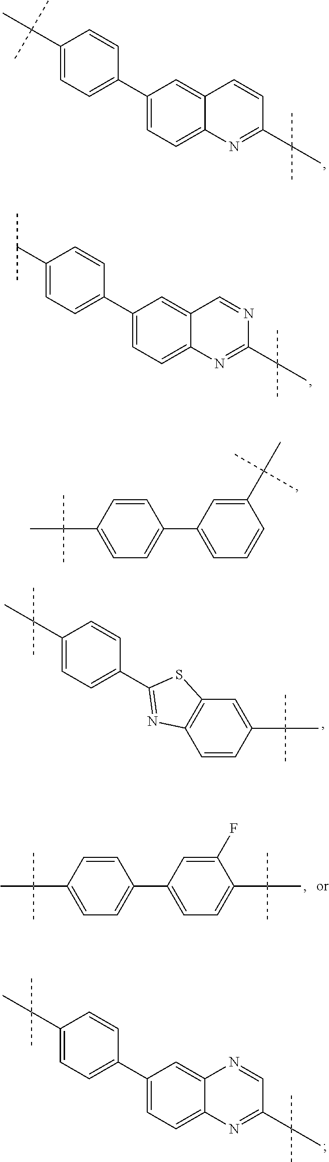

and

### B is

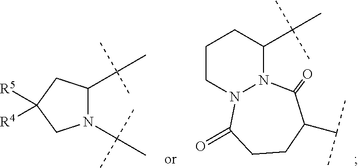

or a pharmaceutically acceptable salt thereof

The application provides a compound of Formula I, wherein B is

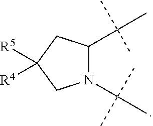

The application provides a compound of Formula I, wherein both R¹ are H, both R² are COOCH₃, and R⁶ is H.

The application provides a compound of Formula I, wherein both R¹ are H, both R² are COOCH₃, R⁶ is H, and B is

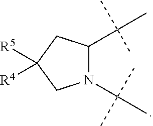

The application provides a compound of Formula I, wherein n is 0.

The application provides a compound of Formula I, wherein n is 0, both R¹ are H, both R² are COOCH₃, R⁶ is H, and B is

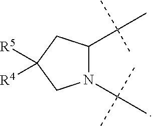

The application provides a compound of Formula I, wherein A is biphenyl.

The application provides a compound of Formula I, wherein A is biphenyl, n is 0, both R¹ are H, both R² are COOCH₃, R⁶ is H, and B is

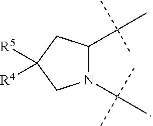

The application provides a compound of Formula I, wherein A is 2-Phenyl-benzothiazole.

The application provides a compound of Formula I, wherein A is 2-Phenyl-benzothiazole, n is 0, both R¹ are H, both R² are COOCH₃, R⁶ is H, and B is

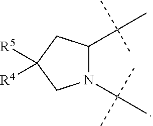

The application provides a compound of Formula I, wherein both R³ are isopropyl.

The application provides a compound of Formula I, wherein both R³ are isopropyl, A is biphenyl, n is 0, both R¹ are H, both R² are COOCH₃, R⁶ is H, and B is

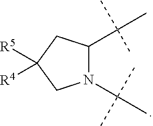

The application provides a compound of Formula I, wherein one R³ is isopropyl and the other is phenyl.

The application provides a compound of Formula I, wherein one R³ is isopropyl and the other is phenyl, A is biphenyl, n is 0, both R¹ are H, both R² are COOCH₃, R⁶ is H, and B is

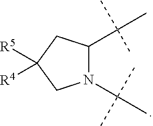

The application provides a compound of Formula I, wherein both R⁴ are H and both R⁵ are H.

The application provides a compound of Formula I, wherein A is biphenyl, n is 0, both R¹ are H, both R² are COOCH₃, R⁶ is H, B is

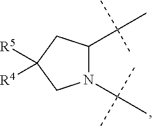

both R⁴ are H, and both R⁵ are H.

The application provides a compound of Formula I, wherein both R⁴ are F and both R⁵ are F.

The application provides a compound of Formula I, wherein both R⁴ and R⁵ are F, A is biphenyl, n is 0, both R¹ are H, both R² are COOCH₃, R⁶ is H, and B is

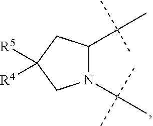

both R⁴ are F, and both R⁵ are F.

The application provides a compound selected from the group consisting of:
((S)-1-{(S)-2-[3-(4′-{2-[(S)-1-(S)-2-Methoxycarbonylamino-3-methyl-butyryl)-pyrrolidin-2-yl]-3H-imidazol-4-yl}-biphenyl-4-yl)-ureidocarbonyl]-pyrrolidine-1-carbonyl}-2-methyl-propyl)-carbamic acid methyl ester;
((S)-1-{(S)-2-[3-(4′-{2-[(S)-1-(S)-2-Methoxycarbonylamino-3-methyl-butyryl)-pyrrolidin-2-yl]-3H-imidazol-4-yl}-biphenyl-3-yl)-ureidocarbonyl]-pyrrolidine-1-carbonyl}-2-methyl-propyl)-carbamic acid methyl ester;
((S)-1-{(S)-2-[5-(4′-{3-[(S)-1-(R)-2-Methoxycarbonylamino-2-phenyl-acetyl)-pyrrolidine-2-carbonyl]-ureido}-biphenyl-4-yl)-1H-imidazol-2-yl]-pyrrolidine-1-carbonyl}-2-methyl-propyl)-carbamic acid methyl ester;
((S)-1-{(S)-2-[5-(4′-{3-[(S)-1-(R)-2-Dimethylamino-2-phenyl-acetyl)-pyrrolidine-2-carbonyl]-ureido}-biphenyl-4-yl)-1H-imidazol-2-yl]-pyrrolidine-1-carbonyl}-2-methyl-propyl)-carbamic acid methyl ester;
((S)-1-{(S)-2-[5-(4′-{3-[(S)-1-(S)-2-Methoxycarbonylamino-3-methyl-butyryl)-pyrrolidine-2-carbonyl]-ureidomethyl}-biphenyl-4-yl)-1H-imidazol-2-yl]-pyrrolidine-1-carbonyl}-2-methyl-propyl)-carbamic acid methyl ester;
((S)-1-{(S)-2-[5-(4′-{3-[(S)-1-(R)-2-Methoxycarbonylamino-2-phenyl-acetyl)-pyrrolidine-2-carbonyl]-ureidomethyl}-biphenyl-4-yl)-1H-imidazol-2-yl]-pyrrolidine-1-carbonyl}-2-methyl-propyl)-carbamic acid methyl ester;
{(4S,7S)-4-[5-(4′-{3-[(S)-1-(R)-2-Methoxycarbonylamino-2-phenyl-acetyl)-pyrrolidine-2-carbonyl]-ureido}-biphenyl-4-yl)-1H-imidazol-2-yl]-6,10-dioxo-octahydro-pyridazino[1,2-a][1,2]diazepin-7-yl}-carbamic acid methyl ester;
(S)-2-{3-[2-(4-{2-[(S)-1-(S)-2-Methoxycarbonylamino-3-methyl-butyryl)-pyrrolidin-2-yl]-3H-imidazol-4-yl}-phenyl)-benzothiazol-6-yl]-ureidocarbonyl}-pyrrolidine-1-carboxylic acid tert-butyl ester;
[(S)-1-((S)-2-{5-[4-(6-{3-[(S)-1-((S)-2-Methoxycarbonylamino-3-methyl-butyryl)-pyrrolidine-2-carbonyl]-ureido}-benzothiazol-2-yl)-phenyl]-1H-imidazol-2-yl}-pyrrolidine-1-carbonyl)-2-methyl-propyl]-carbamic acid methyl ester;
[(S)-1-((S)-2-{5-[4-(5-{3-[(S)-1-(R)-2-Methoxycarbonylamino-2-phenyl-acetyl)-pyrrolidine-2-carbonyl]-ureido}-pyridin-2-yl)-phenyl]-1H-imidazol-2-yl}-pyrrolidine-1-carbonyl)-2-methyl-propyl]-carbamic acid methyl ester;
[(S)-1-((S)-2-{5-[4-(6-{3-[(S)-1-(R)-2-Dimethylamino-2-phenyl-acetyl)-pyrrolidine-2-carbonyl]-ureido}-benzothiazol-2-yl)-phenyl]-1H-imidazol-2-yl}-pyrrolidine-1-carbonyl)-2-methyl-propyl]-carbamic acid methyl ester; and
((S)-1-{(S)-2-[5-(3′-Fluoro-4′-{3-[(S)-1-(S)-2-methoxycarbonylamino-3-methyl-butyryl)-pyrrolidine-2-carbonyl]-ureido}-biphenyl-4-yl)-1H-imidazol-2-yl]-pyrrolidine-1-carbonyl}-2-methyl-propyl)-carbamic acid methyl ester.

The application provides a method for treating a Hepatitis C Virus (HCV) infection comprising administering to a patient in need thereof a therapeutically effective amount of a compound of Formula I.

The application provides the above method, further comprising administering an immune system modulator or an antiviral agent that inhibits replication of HCV, or a combination thereof.

The application provides the above method, wherein the immune system modulator is an interferon or chemically derivatized interferon.

The application provides the above method, wherein the antiviral agent is selected from the group consisting of a HCV protease inhibitor, a HCV polymerase inhibitor, a HCV helicase inhibitor, a HCV primase inhibitor, a HCV fusion inhibitor, and a combination thereof.

The application provides a method for inhibiting replication of HCV in a cell comprising administering a compound of Formula I.

The application provides a composition comprising a compound of Formula I and a pharmaceutically acceptable excipient.

The application provides a method for treating a Hepatitis C Virus (HCV) infection comprising administering to a patient in need thereof a therapeutically effective amount of a compound of Formula I.

The application provides the above method, further comprising administering an immune system modulator or an antiviral agent that inhibits replication of HCV, or a combination thereof.

The application provides the above method, wherein the immune system modulator is an interferon or chemically derivatized interferon.

The application provides the above methods, wherein the antiviral agent is selected from the group consisting of a HCV protease inhibitor, a HCV polymerase inhibitor, a HCV helicase inhibitor, a HCV primase inhibitor, a HCV fusion inhibitor, and a combination thereof.

The application provides a method for inhibiting replication of HCV in a cell comprising administering a compound of Formula I.

The application provides a composition comprising a compound of Formula I and a pharmaceutically acceptable excipient.

### Compounds

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this application is based on AUTONOMTM v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I depicts examples of compounds according to generic Formula I:

TABLE I

| # | Nomenclature | Structure |
|---|---|---|
| I-1 | ((S)-1-{(S)-2-[3-(4′-{2- [(S)-1-((S)-2-Methoxy- carbonylamino-3- methyl-butyryl)- pyrrolidin-2-yl]-3H- imidazol-4-yl}-biphenyl- 4-yl)-ureidocarbonyl]- pyrrolidine-1-carbonyl}- 2-methyl-propyl)- carbamic acid methyl ester | 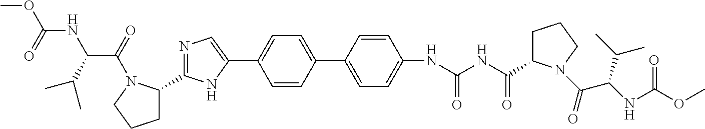 |
| I-2 | ((S)-1-{(S)-2-[3-(4′-{2- [(S)-1-((S)-2-Methoxy- carbonylamino-3- methyl-butyryl)- pyrrolidin-2-yl]-3H- imidazol-4-yl}-biphenyl- 3-yl)-ureidocarbonyl]- pyrrolidine-1-carbonyl}- 2-methyl-propyl)- carbamic acid methyl ester | 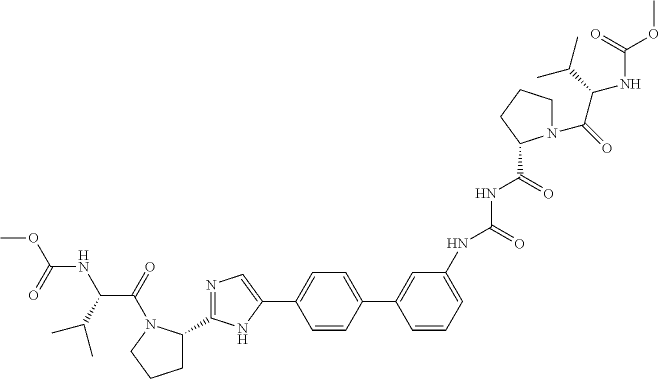 |
| I-3 | ((S)-1-{(S)-2-[5-(4′-{3- [(S)-1-((R)-2-Methoxy- carbonylamino-2- phenyl-acetyl)- pyrrolidine-2-carbonyl]- ureido}-biphenyl-4-yl)- 1H-imidazol-2-yl]- pyrrolidine-1-carbonyl}- 2-methyl-propyl)- carbamic acid methyl ester | 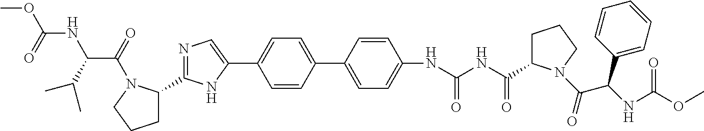 |
| I-4 | ((S)-1-{(S)-2-[5-(4′-{3- [(S)-1-((R)-2-Dimeth- ylamino-2-phenyl- acetyl)-pyrrolidine-2- carbonyl]-ureido}- biphenyl-4-yl)-1H- imidazol-2-yl]- pyrrolidine-1-carbonyl}- 2-methyl-propyl)- carbamic acid methyl ester | 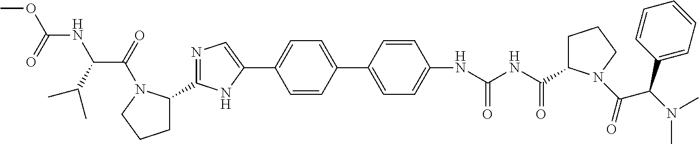 |
| I-5 | ((S)-1-{(S)-2-[5-(4′-{3- [(S)-1-((S)-2-Methoxy- carbonylamino-3- methyl-butyryl)- pyrrolidine-2-carbonyl]- ureidomethyl}-biphenyl- 4-yl)-1H-imidazol-2-yl]- pyrrolidine-1-carbonyl}- 2-methyl-propyl)- carbamic acid methyl ester | 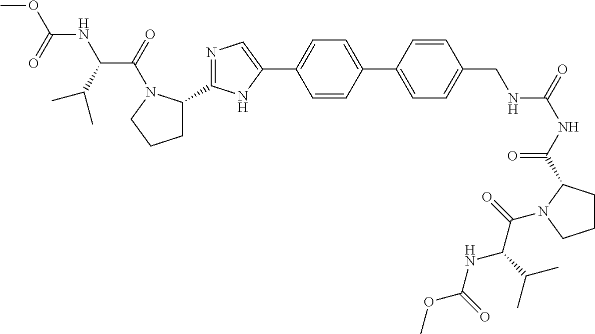 |
| I-6 | ((S)-1-{(S)-2-[5-(4′-{3- [(S)-1-((R)-2- Methoxycarbonylamino- 2-phenyl-acetyl)- pyrrolidine-2-carbonyl]- ureidomethyl}-biphenyl- 4-yl)-1H-imidazol-2-yl]- pyrrolidine-1-carbonyl}- 2-methyl-propyl)- carbamic acid methyl ester | 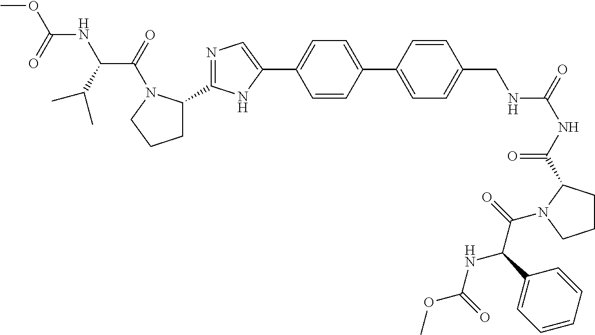 |
| I-7 | {(4S,7S)-4-[5-(4′-{3- [(S)-1-((R)-2- Methoxycarbonylamino- 2-phenyl-acetyl)- pyrrolidine-2-carbonyl]- ureido}-biphenyl-4-yl)- 1H-imidazol-2-yl]-6,10- dioxo-octahydro- pyridazino[1,2- a][1,2]diazepin-7-yl}- carbamic acid methyl ester | 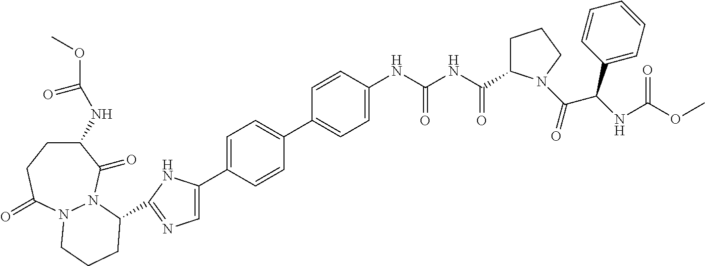 |
| I-8 | (S)-2-{3-[2-(4-{2-[(S)-1- ((S)-2-Methoxy- carbonylamino-3- methyl-butyryl)- pyrrolidin-2-yl]-3H- imidazol-4-yl}-phenyl)- benzothiazol-6-yl]- ureidocarbonyl}- pyrrolidine-1-carboxylic acid tert-butyl ester | 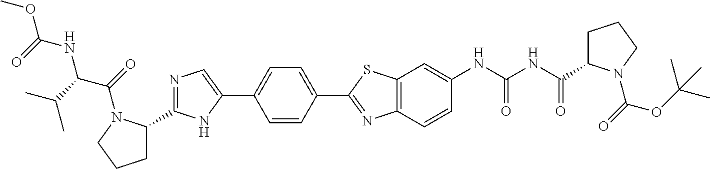 |
| I-9 | [(S)-1-((S)-2-{5-[4-(6-{3- [(S)-1-((S)-2- Methoxycarbonylamino- 3-methyl-butyryl)- pyrrolidine-2-carbonyl]- ureido}-benzothiazol-2- yl)-phenyl]-1H-imi- dazol-2-yl}-pyrrolidine- 1-carbonyl)-2-methyl- propyl]-carbamic acid methyl ester | 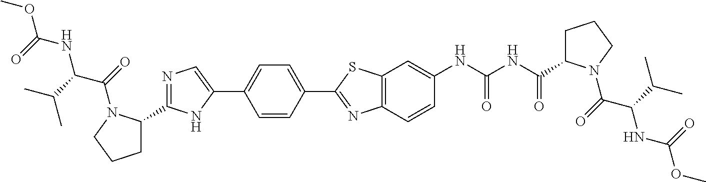 |
| I-10 | [(S)-1-((S)-2-{5-[4-(5- {3-[(S)-1-((R)-2- Methoxycarbonylamino- 2-phenyl-acetyl)- pyrrolidine-2-carbonyl]- ureido}-pyridin-2-yl)- phenyl]-1H-imidazol-2- yl}-pyrrolidine-1- carbonyl)-2-methyl- propyl]-carbamic acid methyl ester | 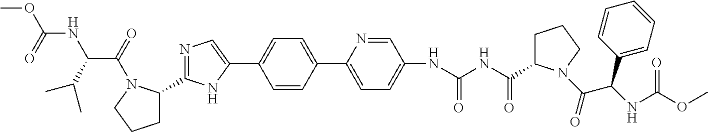 |
| I-11 | [(S)-1-((S)-2-{5-[4-(6- {3-[(S)-1-((R)-2-Di- methylamino-2-phenyl- acetyl)-pyrrolidine-2- carbonyl]-ureido}- benzothiazol-2-yl)- phenyl]-1H-imidazol-2- yl}-pyrrolidine-1- carbonyl)-2-methyl- propyl]-carbamic acid methyl ester | 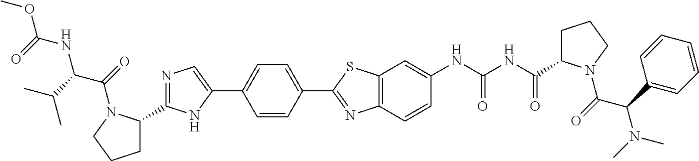 |
| I-12 | ((S)-1-{(S)-2-[5-(3′- Fluoro-4′-{3-[(S)-1- ((S)-2-methoxy- carbonylamino-3- methyl-butyryl)- pyrrolidine-2-carbonyl]- ureido}-biphenyl-4-yl)- 1H-imidazol-2-yl]- pyrrolidine-1-carbonyl}- 2-methyl-propyl)- carbamic acid methyl ester | 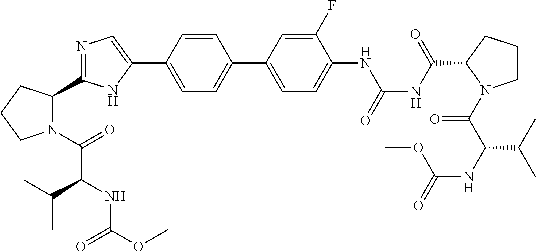 |

### Synthesis

### General Schemes

The following schemes depict general methods for obtaining compounds of Formula I:

Compounds of formula I can be prepared following the general scheme 1. Compounds of formula II and III can be reacted together using Pd⁰-coupling methods to yield compounds of formula IV. Compounds of formula IV can be N-deprotected using appropriate method and the resulting amine can be coupled with the corresponding amino acid derivatives to yield compounds of formula I.

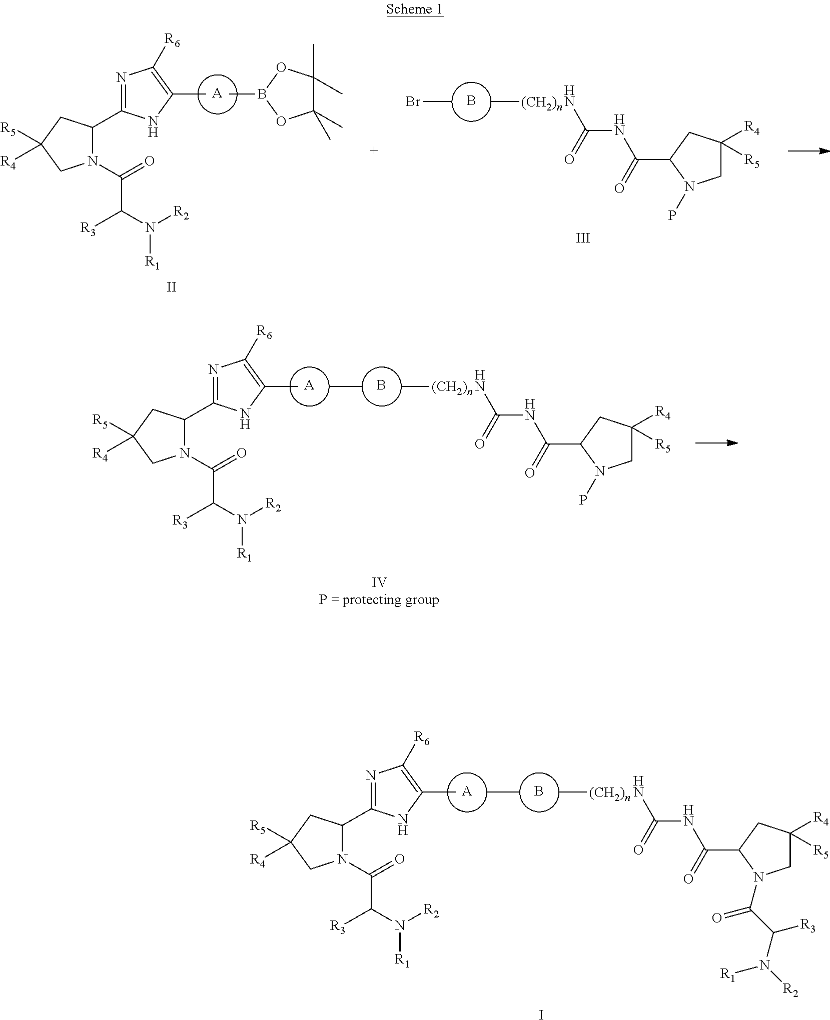

Compounds of formula II are readily accessible following methods disclosed in earlier reports (see for example, WO2008/0311075). Compounds for formula III are accessible following reaction sequence shown in Scheme 2. The isocyanates of formula IV are either commercially available or readily prepared from the corresponding amines via the well known methods (for example, reactions between amines and phosgene, see Ozaki et al, Chemical Reviews, 72, 457-496, 1972, for reactions using Curtius rearrangement, see Banthorpe et al, in Patai, “The Chemistry of the azido group, pp, 397-405, Interscience, New York, 1971), which can be used to react with carboxamides of formula V to yield N-acyl ureas of formula III (see for example, Wiley, P, Journal of the American Chemical Society, 71, 1310-11, 1949 or see Bandurco, V et al, Journal of Medicinal Chemistry 30, 1421-6, 1987).

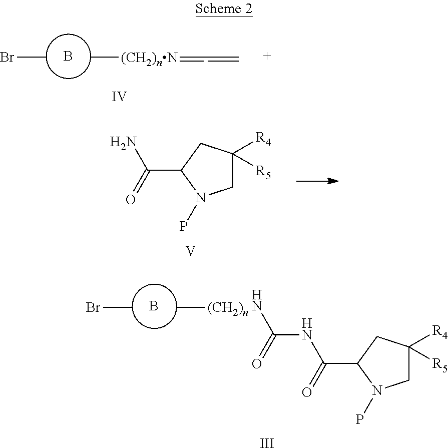

Compounds of formula V are readily available from the corresponding protected proline derivatives, via amination reaction of the corresponding acid chlorides. The proline derivatives are commercially available or can be prepared following the reported methods.

### Pharmaceutical Compositions and Administration

Pharmaceutical compositions of the subject Compounds for administration via several routes were prepared as described in this Example.

### Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

### Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

### Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |

| | |
|---|---|
| Distilled water | q.s. to 100 ml |

The ingredients are mixed to form a suspension for oral administration.

### Parenteral Formulation (D)

| Ingredient | % wt./wt. | |
|---|---|---|
| Active ingredient | 0.25 | g |
| Sodium Chloride | qs to make isotonic | |
| Water for injection to | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

### Dosage and Administration:

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polylactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Indications and Method of Treatment

### Indications

The compounds of the invention and their isomeric forms and pharmaceutically acceptable salts thereof are useful in treating and preventing HCV infection.

The application provides a method for treating a Hepatitis C Virus (HCV) infection comprising administering to a patient in need thereof a therapeutically effective amount of a compound of Formula I.

The application provides a method for inhibiting replication of HCV in a cell comprising administering a compound of Formula I.

### Combination Therapy

The compounds of the invention and their isomeric forms and pharmaceutically acceptable salts thereof are useful in treating and preventing HCV infection alone or when used in combination with other compounds targeting viral or cellular elements or functions involved in the HCV lifecycle. Classes of compounds useful in the invention include, without limitation, all classes of HCV antivirals.

For combination therapies, mechanistic classes of agents that can be useful when combined with the compounds of the invention include, for example, nucleoside and non-nucleoside inhibitors of the HCV polymerase, protease inhibitors, helicase inhibitors, NS4B inhibitors and medicinal agents that functionally inhibit the internal ribosomal entry site (IRES) and other medicaments that inhibit HCV cell attachment or virus entry, HCV RNA translation, HCV RNA transcription, replication or HCV maturation, assembly or virus release. Specific compounds in these classes and useful in the invention include, but are not limited to, macrocyclic, heterocyclic and linear HCV protease inhibitors such as telaprevir (VX-950), boceprevir (SCH-503034), narlaprevir (SCH-9005 18), ITMN-191 (R-7227), TMC-435350 (a.k.a. TMC-435), MK-7009, BI-201335, BI-2061 (ciluprevir), BMS-650032, ACH-1625, ACH-1095 (HCV NS4A protease co-factor inhibitor), VX-500, VX-8 13, PHX-1766, PHX2054, IDX-136, IDX-3 16, ABT-450 EP-0 13420 (and congeners) and VBY-376; the Nucleosidic HCV polymerase (replicase) inhibitors useful in the invention include, but are not limited to, R7128, PSI-785 1, IDX-184, IDX-102, R1479, UNX-08 189, PSI-6130, PSI-938 and PSI-879 and various other nucleoside and nucleotide analogs and HCV inhibitors including (but not limited to) those derived as 2′-C-methyl modified nucleos(t)ides, 4′-aza modified nucleos(t)ides, and 7′-deaza modified nucleos(t)ides. Non-nucleosidic HCV polymerase (replicase) inhibitors useful in the invention, include, but are not limited to, HCV-796, HCV-371, VCH-759, VCH-916, VCH-222, ANA-598, MK-3281, ABT-333, ABT-072, PF-00868554, BI-207127, GS-9190, A-837093, JKT-109, GL-59728 and GL-60667.

In addition, compounds of the invention can be used in combination with cyclophyllin and immunophyllin antagonists (e.g., without limitation, DEBIO compounds, NM-811 as well as cyclosporine and its derivatives), kinase inhibitors, inhibitors of heat shock proteins (e.g., HSP90 and HSP70), other immunomodulatory agents that can include, without limitation, interferons (-alpha, -beta, -omega, -gamma, -lambda or synthetic) such as Intron A, Roferon-A, Canferon-A300, Advaferon, Infergen, Humoferon, Sumiferon MP, Alfaferone, IFN-β, Feron and the like; polyethylene glycol derivatized (pegylated) interferon compounds, such as PEG interferon-α-2a (Pegasys), PEG interferon-α-2b (PEGIntron), pegylated IFN-α-con1 and the like; long acting formulations and derivatizations of interferon compounds such as the albumin-fused interferon, Albuferon, Locteron, and the like; interferons with various types of controlled delivery systems (e.g., ITCA-638, omega-interferon delivered by the DUROS subcutaneous delivery system); compounds that stimulate the synthesis of interferon in cells, such as resiquimod and the like; interleukins; compounds that enhance the development of type 1 helper T cell response, such as SCV-07 and the like; TOLL-like receptor agonists such as CpG-10101 (actilon), isotorabine, ANA773 and the like; thymosin α-1; ANA-245 and ANA-246; histamine dihydrochloride; propagermanium; tetrachlorodecaoxide; ampligen; IMP-321; KRN-7000; antibodies, such as civacir, XTL-6865 and the like and prophylactic and therapeutic vaccines such as InnoVac C, HCV E1E2/MF59 and the like. In addition, any of the above-described methods involving administering an NS5A inhibitor, a Type I interferon receptor agonist (e.g., an IFN-α) and a Type II interferon receptor agonist (e.g., an IFN-γ) can be augmented by administration of an effective amount of a TNF-α antagonist. Exemplary, non-limiting TNF-α antagonists that are suitable for use in such combination therapies include ENBREL, REMICADE, and HUMIRA.

In addition, compounds of the invention can be used in combination with antiprotozoans and other antivirals thought to be effective in the treatment of HCV infection such as, without limitation, the prodrug nitazoxanide. Nitazoxanide can be used as an agent in combination with the compounds disclosed in this invention as well as in combination with other agents useful in treating HCV infection such as peginterferon α-2a and ribavirin.

Compounds of the invention can also be used with alternative forms of interferons and pegylated interferons, ribavirin or its analogs (e.g., tarabavarin, levoviron), microRNA, small interfering RNA compounds (e.g., SIRPLEX-140-N and the like), nucleotide or nucleoside analogs, immunoglobulins, hepatoprotectants, anti-inflammatory agents and other inhibitors of NS5A. Inhibitors of other targets in the HCV lifecycle include NS3 helicase inhibitors; NS4A co-factor inhibitors; antisense oligonucleotide inhibitors, such as ISIS-14803, AVI-4065 and the like; vector-encoded short hairpin RNA (shRNA); HCV specific ribozymes such as heptazyme, RPI, 13919 and the like; entry inhibitors such as HepeX-C, HuMax-HepC and the like; alpha glucosidase inhibitors such as celgosivir, UT-231B and the like; KPE-02003002 and BIVN 401 and IMPDH inhibitors. Other illustrative HCV inhibitor compounds include those disclosed in the following publications: U.S. Pat. Nos. 5,807,876; 6,498,178; 6,344,465; and 6,054,472; PCT Patent Application Publication Nos. WO97/40028; WO98/40381; WO00/56331, WO02/04425; WO03/007945; WO03/010141; WO03/000254; WO01/32153; WO00/06529; WO00/18231; WO00/10573; WO00/13708; WO01/85172; WO03/037893; WO03/037894; WO03/037895; WO02/100851; WO02/100846; WO99/01582; WO00/09543; WO02/18369; WO98/17679, WO00/056331; WO98/22496; WO99/07734; WO05/073216, WO05/073195 and WO08/021,927.

Additionally, combinations of, for example, ribavirin and interferon, may be administered as multiple combination therapy with at least one of the compounds of the invention. The present invention is not limited to the aforementioned classes or compounds and contemplates known and new compounds and combinations of biologically active agents. It is intended that combination therapies of the present invention include any chemically compatible combination of a compound of this inventive group with other compounds of the inventive group or other compounds outside of the inventive group, as long as the combination does not eliminate the anti-viral activity of the compound of this inventive group or the anti-viral activity of the pharmaceutical composition itself.

Combination therapy can be sequential, that is treatment with one agent first and then a second agent (for example, where each treatment comprises a different compound of the invention or where one treatment comprises a compound of the invention and the other comprises one or more biologically active agents) or it can be treatment with both agents at the same time (concurrently). Sequential therapy can include a reasonable time after the completion of the first therapy before beginning the second therapy. Treatment with both agents at the same time can be in the same daily dose or in separate doses. Combination therapy need not be limited to two agents and may include three or more agents. The dosages for both concurrent and sequential combination therapy will depend on absorption, distribution, metabolism and excretion rates of the components of the combination therapy as well as other factors known to one of skill in the art. Dosage values will also vary with the severity of the condition to be alleviated. It is to be further understood that for any particular subject, specific dosage regimens and schedules may be adjusted over time according to the individual's need and the judgment of the one skilled in the art administering or supervising the administration of the combination therapy.

The application provides a method for treating a Hepatitis C Virus (HCV) infection comprising administering to a patient in need thereof a therapeutically effective amount of a compound of Formula I.

The application provides the above method, further comprising administering an immune system modulator or an antiviral agent that inhibits replication of HCV, or a combination thereof.

The application provides the above method, wherein the immune system modulator is an interferon or chemically derivatized interferon.

The application provides the above methods, wherein the antiviral agent is selected from the group consisting of a HCV protease inhibitor, a HCV polymerase inhibitor, a HCV helicase inhibitor, a HCV primase inhibitor, a HCV fusion inhibitor, and a combination thereof.

### EXAMPLES

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), 2,2′-bis(diphenylphosphino)-1,1′-binaphthyl (BINAP), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), 2,3-Dichloro-5,6-dicyano-1,4-benzoquinone (DDQ), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), 2-ethoxy-1-ethoxycarbonyl-1,2-dihydroquinoline (EEDQ), ethyl (Et), ethyl acetate (EtOAc), ethanol (EtOH), 2-ethoxy-2H-quinoline-1-carboxylic acid ethyl ester (EEDQ), diethyl ether (Et₂O), ethyl isopropyl ether (EtOiPr), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), isopropylmagnesium chloride (iPrMgCl), hexamethyl disilazane (HMDS), liquid chromatography mass spectrometry (LCMS), lithium hexamethyl disilazane (LiHMDS), meta-chloroperoxybenzoic acid (m-CPBA), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), methyl tetrahydrofuran (MeTHF), N-bromosuccinimide (NBS), n-Butyllithium (nBuLi), N-carboxyanhydride (NCA), N-chlorosuccinimide (NCS), N-methylmorpholine (NMM), N-methylpyrrolidone (NMP), pyridinium chlorochromate (PCC), Dichloro-((bis-diphenylphosphino)ferrocenyl)palladium(II) (Pd(dppf)Cl₂), palladium(II)acetate (Pd(OAc)₂), tris(dibenzylideneacetone)dipalladium(0) (Pd₂(dba)₃), pyridinium dichromate (PDC), phenyl (Ph), propyl (Pr), iso-propyl (i-Pr), pounds per square inch (psi), pyridine (pyr), 1,2,3,4,5-Pentaphenyl-1′-(di-tert-butylphosphino)ferrocene (Q-Phos), room temperature (ambient temperature, rt or RT), sec-Butyllithium (sBuLi), tert-butyldimethylsilyl or t-BuMe₂Si (TBDMS), tetra-n-butylammonium fluoride (TBAF), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), and N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### General Conditions

Compounds of the invention can be made by a variety of methods depicted in the illustrative synthetic reactions described below in the Examples section.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

### Preparative Examples

### Example 1

### ((S)-1-{(S)-2-[3-(4′-{2-[(S)-1-((S)-2-Methoxycarbonylamino-3-methyl-butyryl)-pyrrolidin-2-yl]-3H-imidazol-4-yl}-biphenyl-4-yl)-ureidocarbonyl]-pyrrolidine-1-carbonyl}-2-methyl-propyl)-carbamic acid methyl ester

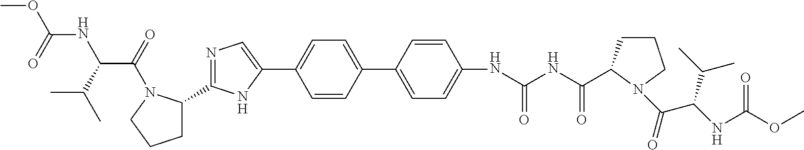

A mixture of (S)-tert-butyl 2-carbamoylpyrrolidine-1-carboxylate (1.0 g, 4.67 mmol) and 1-bromo-4-isocyanatobenzene (924 mg, 4.67 mmol) in toluene (10 mL) was stirred at reflux for 3 h. Upon completion of the reaction the solid was removed by filtration and the filtrate concentrated. The crude product obtained was purified by ISCO flash chromatography (Teledyne Isco RediSep Flash Column 40 g; (0% to 100% ethyl acetate/hexane) to afford, (S)-2-[3-(4-bromo-phenyl)-ureidocarbonyl]-pyrrolidine-1-carboxylic acid tert-butyl ester as a white solid, (1.30 g, 68%): ESI-LRMS m/e calcd for C₁₇H₂₂BrN₃O₄ [M⁺] 412. found 413 [M+H⁺].

To a stirred mixture of (S)-2-[3-(4-bromo-phenyl)-ureidocarbonyl]-pyrrolidine-1-carboxylic acid tert-butyl ester (1.30 g, 3.15 mmol) dissolved in methanol (20 mL) was added a 4.0M HCl/dioxane solution (10 mL). After addition was complete the mixture was stirred at room temperature for 4 h. The reaction mixture was then concentrated in vacuo to afford, 1-(4-bromo-phenyl)-3-(S)-pyrrolidine-2-carbonyl)-urea hydrochloride as a white powder, (1.08 g, 98%): ESI-LRMS m/e calcd for C₁₂H₁₄BrN₃O₂₋HCl [M⁺] 348.5. found 313 [M+H⁺] (free base).

N,N′-Diisopropylethylamine (556 mg, 4.30 mmol) was added to a solution of 1-(4-bromo-phenyl)-3-(S)-pyrrolidine-2-carbonyl)-urea hydrochloride (500 mg, 1.43 mmol), (S)-valine t-butyl ester (251 mg, 1.43 mmol) and HATU (545 mg, 1.43 mmol) in DMF (10 mL). After the addition was complete the reaction was stirred at room temperature for 4 h. The reaction was diluted with ethyl acetate and washed with water, 2N hydrochloric acid, a saturated sodium bicarbonate solution, a saturated sodium chloride solution and dried over magnesium sulfate, filtered and concentrated to afford, ((S)-1-{(S)-2-[3-(4-Bromo-phenyl)-ureidocarbonyl]-pyrrolidine-1-carbonyl}-2-methyl-propyl)-carbamic acid methyl ester as a white powder, (671 mg, 99%): ESI-LRMS m/e calcd for C₁₉H₂₅BrN₄O₅ [M⁺] 469. found 470 [M+H⁺].

In a sealed tube tetrakis(triphenylphosphine)palladium(0) (47 mg, 0.04 mmol) was added to a mixture of methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (200 mg, 0.40 mmol) (BMS patent), ((S)-1-{(S)-2-[3-(4-bromo-phenyl)-ureidocarbonyl]-pyrrolidine-1-carbonyl}-2-methyl-propyl)-carbamic acid methyl ester (189 mg, 0.40 mmol) and sodium bicarbonate (102 mg, 1.21 mmol) in 1,2-dimethoxyethane (12 mL) and water (4 mL). The reaction mixture was flushed with nitrogen, capped and heated to 80° C. for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, ((S)-1-{(S)-2-[3-(4′-{2-[(S)-1-((S)-2-methoxycarbonylamino-3-methyl-butyryl)-pyrrolidin-2-yl]-3H-imidazol-4-yl}-biphenyl-4-yl)-ureidocarbonyl]-pyrrolidine-1-carbonyl}-2-methyl-propyl)-carbamic acid methyl ester as a white solid, (43 mg, 14%): ESI-LRMS m/e calcd for C₃₉H₅₀N₈O₈ [M⁺] 758. found 759. ¹H NMR (400 MHz, DMSO-d₆) δ ppm 0.80-0.98 (m, 12H) 1.18-1.30 (m, 2H) 1.84-2.04 (m, 5H) 2.09-2.24 (m, 2H) 3.53 (d, J=3.01 Hz, 6H) 3.59-3.68 (m, 1H) 3.81 (d, J=5.77 Hz, 2H) 4.04 (dt, J=14.24, 8.44 Hz, 2H) 4.47 (br. s., 1H) 5.07 (dd, J=6.90, 3.14 Hz, 1H) 7.31 (d, J=8.28 Hz, 1H) 7.43 (d, J=8.28 Hz, 1H) 7.51 (d, J=1.76 Hz, 1H) 7.56-7.70 (m, 8H) 7.78 (d, J=8.28 Hz, 2H) 10.44 (br. s., 1H) 10.92 (br. s., 1H) 11.78 (s, 1H).

### Example 2

### ((S)-1-{(S)-2-[3-(4′-{2-[(S)-1-((S)-2-Methoxycarbonylamino-3-methyl-butyryl)-pyrrolidin-2-yl]-3H-imidazol-4-yl}-biphenyl-3-yl)-ureidocarbonyl]-pyrrolidine-1-carbonyl}-2-methyl-propyl)-carbamic acid methyl ester

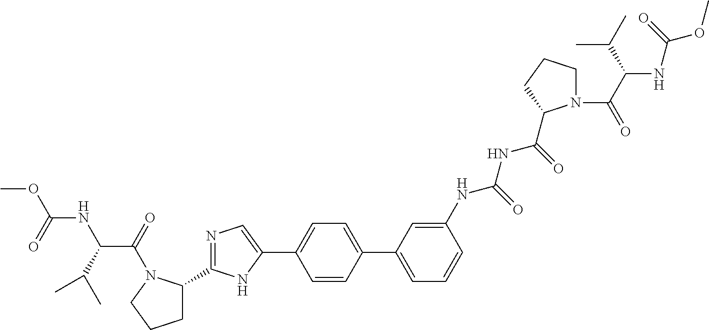

A mixture of (S)-tert-butyl 2-carbamoylpyrrolidine-1-carboxylate (1.0 g, 4.67 mmol) and 1-bromo-3-isocyanatobenzene (924 mg, 4.67 mmol) in toluene (10 mL) was stirred at reflux for 3 h. Upon completion of the reaction the solid was removed by filtration and the filtrate concentrated. The crude product obtained was purified by ISCO flash chromatography (Teledyne Isco RediSep Flash Column 40 g; (0% to 100% ethyl acetate/hexane) to afford, (S)-2-[3-(3-bromo-phenyl)-ureidocarbonyl]-pyrrolidine-1-carboxylic acid tert-butyl ester as a white solid, (1.46 g, 76%): ESI-LRMS m/e calcd for C₁₇H₂₂BrN₃O₄ [M⁺] 412. found 413 [M+H⁺].

To a stirred mixture of (S)-2-[3-(3-bromo-phenyl)-ureidocarbonyl]-pyrrolidine-1-carboxylic acid tert-butyl ester (1.46 g, 3.54 mmol) dissolved in methanol (20 mL) was added a 4.0M HCl/dioxane solution (10 mL). After addition was complete the mixture was stirred at room temperature for 4 h. The reaction mixture was then concentrated in vacuo to afford, 1-(3-bromo-phenyl)-3-(S)-pyrrolidine-2-carbonyl)-urea hydrochloride as a white powder, (1.22 g, 99%): ESI-LRMS m/e calcd for C₁₂H₁₄BrN₃O₂₋HCl [M⁺] 348.5. found 313 [M+H⁺] (free base).

N,N′-Diisopropylethylamine (556 mg, 4.30 mmol) was added to a solution of 1-(3-bromo-phenyl)-3-(S)-pyrrolidine-2-carbonyl)-urea hydrochloride (500 mg, 1.43 mmol), (S)-valine t-butyl ester (251 mg, 1.43 mmol) and HATU (545 mg, 1.43 mmol) in DMF (10 mL). After the addition was complete the reaction was stirred at room temperature for 4 h. The reaction was diluted with ethyl acetate and washed with water, 2N hydrochloric acid, a saturated sodium bicarbonate solution, a saturated sodium chloride solution and dried over magnesium sulfate, filtered and concentrated to afford, ((S)-1-{(S)-2-[3-(3-Bromo-phenyl)-ureidocarbonyl]-pyrrolidine-1-carbonyl}-2-methyl-propyl)-carbamic acid methyl ester as a white powder, (576 mg, 86%): ESI-LRMS m/e calcd for C₁₉H₂₅BrN₄O₅ [M⁺] 469. found 470 [M+H⁺].

In a sealed tube tetrakis(triphenylphosphine)palladium(0) (47 mg, 0.04 mmol) was added to a mixture of methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (200 mg, 0.40 mmol) (BMS patent), ((S)-1-{(S)-2-[3-(3-bromo-phenyl)-ureidocarbonyl]-pyrrolidine-1-carbonyl}-2-methyl-propyl)-carbamic acid methyl ester (189 mg, 0.40 mmol) and sodium bicarbonate (102 mg, 1.21 mmol) in 1,2-dimethoxyethane (12 mL) and water (4 mL). The reaction mixture was flushed with nitrogen, capped and heated to 80° C. for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, ((S)-1-{(S)-2-[3-(4′-{2-[(S)-1-((S)-2-methoxycarbonylamino-3-methyl-butyryl)-pyrrolidin-2-yl]-3H-imidazol-4-yl}-biphenyl-3-yl)-ureidocarbonyl]-pyrrolidine-1-carbonyl}-2-methyl-propyl)-carbamic acid methyl ester as a white solid, (8 mg, 3%): ESI-LRMS m/e calcd for C₃₉H₅₀N₈O₈ [M⁺] 758. found 759 [M+H⁺]; ¹H NMR (400 MHz, DMSO-d₆) δ ppm 0.78-0.98 (m, 12H) 1.24 (d, J=2.26 Hz, 1H) 1.85-2.05 (m, 6H) 2.09-2.23 (m, 2H) 3.53 (d, J=3.51 Hz, 6H) 3.60-3.67 (m, 1H) 3.77-3.87 (m, 2H) 3.98-4.10 (m, 2H) 4.49 (br. s., 1H) 5.08 (dd, J=6.90, 3.39 Hz, 1H) 7.31 (d, J=8.28 Hz, 1H) 7.38-7.45 (m, 2H) 7.52 (d, J=2.01 Hz, 2H) 7.63 (s, 2H) 7.70 (s, 1 H) 7.78 (m, 5H) 10.43 (br. s., 1H) 10.89 (br. s., 1H) 11.80 (s, 1H).

### Example 3

### ((S)-1-{(S)-2-[5-(4′-{3-[(S)-1-[((R)-2-Methoxycarbonylamino-2-phenyl-acetyl)-pyrrolidine-2-carbonyl]-ureido}-biphenyl-4-yl)-1H-imidazol-2-yl]-pyrrolidine-1-carbonyl}-2-methyl-propyl)-carbamic acid methyl ester

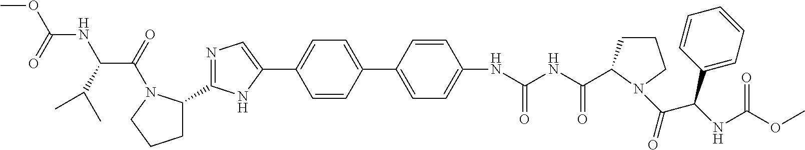

A mixture of (S)-tert-butyl 2-carbamoylpyrrolidine-1-carboxylate (1.0 g, 4.67 mmol) and 1-bromo-4-isocyanatobenzene (924 mg, 4.67 mmol) in toluene (10 mL) was stirred at reflux for 3 h. Upon completion of the reaction the solid was removed by filtration and the filtrate concentrated. The crude product obtained was purified by ISCO flash chromatography (Teledyne Isco RediSep Flash Column 40 g; (0% to 100% ethyl acetate/hexane) to afford, (S)-2-[3-(4-bromo-phenyl)-ureidocarbonyl]-pyrrolidine-1-carboxylic acid tert-butyl ester as a white solid, (1.30 g, 68%): ESI-LRMS m/e calcd for C₁₇H₂₂BrN₃O₄ [M⁺] 412. found 413 [M+H⁺].

To a stirred mixture of (S)-2-[3-(4-bromo-phenyl)-ureidocarbonyl]-pyrrolidine-1-carboxylic acid tert-butyl ester (1.30 g, 3.15 mmol) dissolved in methanol (20 mL) was added a 4.0M HCl/dioxane solution (10 mL). After addition was complete the mixture was stirred at room temperature for 4 h. The reaction mixture was then concentrated in vacuo to afford, 1-(4-bromo-phenyl)-3-(S)-pyrrolidine-2-carbonyl)-urea hydrochloride as a white powder, (1.08 g, 98%): ESI-LRMS m/e calcd for C₁₂H₁₄BrN₃O₂₋HCl [M⁺] 348.5. found 313 [M+H⁺] (free base).

N,N′-Diisopropylethylamine (556 mg, 4.30 mmol) was added to a solution of 1-(4-bromo-phenyl)-3-(S)-pyrrolidine-2-carbonyl)-urea hydrochloride (500 mg, 1.43 mmol), (R)-2-(methoxycarbonylamino)-2-phenylacetic acid (BMS patent) (300 mg, 1.43 mmol) and HATU (545 mg, 1.43 mmol) in DMF (10 mL). After the addition was complete the reaction was stirred at room temperature for 4 h. The reaction was diluted with ethyl acetate and washed with water, 2N hydrochloric acid, a saturated sodium bicarbonate solution, a saturated sodium chloride solution and dried over magnesium sulfate, filtered and concentrated to afford, ((R)-2-{(S)-2-[3-(4-Bromo-phenyl)-ureidocarbonyl]-pyrrolidin-1-yl}-2-oxo-1-phenyl-ethyl)-carbamic acid methyl ester as a clear colorless oil, (448 mg, 62%): ESI-LRMS m/e calcd for C₂₂H₂₃BrN₄O₅ [M⁺] 503. found 531 [M+H⁺].

In a sealed tube tetrakis(triphenylphosphine)palladium(0) (23 mg, 0.02 mmol) was added to a mixture of methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (99 mg, 0.40 mmol) (BMS patent), ((R)-2-{(S)-2-[3-(4-Bromo-phenyl)-ureidocarbonyl]-pyrrolidin-1-yl}-2-oxo-1-phenyl-ethyl)-carbamic acid methyl ester (100 mg, 0.20 mmol) and sodium bicarbonate (50 mg, 0.60 mmol) in 1,2-dimethoxyethane (6 mL) and water (1 mL). The reaction mixture was flushed with nitrogen, capped and heated to 80° C. for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, ((S)-1-{(S)-2-[5-(4′-{3-[(S)-1-(R)-2-Methoxycarbonylamino-2-phenyl-acetyl)-pyrrolidine-2-carbonyl]-ureido}-biphenyl-4-yl)-1H-imidazol-2-yl}-pyrrolidine-1-carbonyl}-2-methyl-propyl)-carbamic acid methyl ester as a white solid, (9 mg, 6%): ESI-LRMS m/e calcd for C₄₂H₄₈N₈O₈ [M⁺] 792. found 793 [M+H⁺]; ¹H NMR (400 MHz, DMSO-d₆) δ ppm 0.88-0.99 (m, 6H) 1.94-2.12 (m, 6H) 2.36-2.41 (m, 3H) 2.74 (t, J=1.76 Hz, 1H) 3.60 (s, 6H) 3.87 (br. s., 2H) 4.08-4.16 (m, 1H) 4.50 (d, J=5.27 Hz, 1H) 5.14 (d, J=3.26 Hz, 2H) 5.59 (d, J=8.28 Hz, 1H) 7.23-7.88 (m, 15H) 8.60 (s, 1H) 10.58 (br. s., 1H) 10.97 (br. s., 1H) 11.85 (s, 1H).

### Example 4

### Methyl (S)-1-((S)-2-(5-(4′-(3-((S)-1-OR)-2-(dimethylamino)-2-phenylacetyl)pyrrolidine-2-carbonyl)ureido)biphenyl-4-yl)-1H-imidazol-2-yl)pyrrolidin-1-yl)-3-methyl-1-oxobutan-2-ylcarbamate

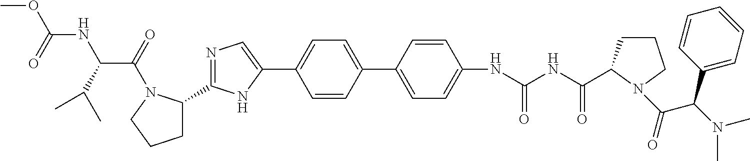

A mixture of (S)-tert-butyl 2-carbamoylpyrrolidine-1-carboxylate (1.0 g, 4.67 mmol) and 1-bromo-4-isocyanatobenzene (924 mg, 4.67 mmol) in toluene (10 mL) was stirred at reflux for 3 h. Upon completion of the reaction the solid was removed by filtration and the filtrate concentrated. The crude product obtained was purified by ISCO flash chromatography (Teledyne Isco RediSep Flash Column 40 g; (0% to 100% ethyl acetate/hexane) to afford, (S)-2-[3-(4-bromo-phenyl)-ureidocarbonyl]-pyrrolidine-1-carboxylic acid tert-butyl ester as a white solid, (1.30 g, 68%): ESI-LRMS m/e calcd for C₁₇H₂₂BrN₃O₄ [M⁺] 412. found 413 [M+H⁺].

To a stirred mixture of (S)-2-[3-(4-bromo-phenyl)-ureidocarbonyl]-pyrrolidine-1-carboxylic acid tert-butyl ester (1.30 g, 3.15 mmol) dissolved in methanol (20 mL) was added a 4.0M HCl/dioxane solution (10 mL). After addition was complete the mixture was stirred at room temperature for 4 h. The reaction mixture was then concentrated in vacuo to afford, 1-(4-bromo-phenyl)-3-(S)-pyrrolidine-2-carbonyl)-urea hydrochloride as a white powder, (1.08 g, 98%): ESI-LRMS m/e calcd for C₁₂H₁₄BrN₃O₂₋HCl [M⁺] 348.5. found 313 [M+H⁺] (free base).

N,N′-Diisopropylethylamine (445 mg, 3.44 mmol) was added to a solution of 1-(4-bromo-phenyl)-3-(S)-pyrrolidine-2-carbonyl)-urea hydrochloride (300 mg, 0.86 mmol), (R)-dimethylamino-phenylacetic acid hydrochloride (BMS patent) (186 mg, 0.86 mmol) and HATU (327 mg, 0.86 mmol) in DMF (10 mL). After the addition was complete the reaction was stirred at room temperature for 4 h. The reaction was diluted with ethyl acetate and washed with water, a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (0% to 100% ethyl acetate/hexane to afford, 1-(4-bromo-phenyl)-3-[(S)-1-(R)-2-dimethylamino-2-phenyl-acetyl)-pyrrolidine-2-carbonyl]-urea as a white solid, (252 mg, 62%): ESI-LRMS m/e calcd for C₂₂H₂₅BrN₄O₃ [M⁺] 473. found 474 [M+H⁺].

In a sealed tube [1,1′-bis(diphenylphosphino)-ferrocene]dichloropalladium(II) (39 mg, 0.05 mmol) was added to a mixture of methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (262 mg, 0.53 mmol) (BMS patent), 1-(4-bromo-phenyl)-3-[(S)-1-(R)-2-dimethylamino-2-phenyl-acetyl)-pyrrolidine-2-carbonyl]-urea (250 mg, 0.53 mmol) and sodium bicarbonate (133 mg, 1.58 mmol) in 1,2-dimethoxyethane (6 mL) and water (1 mL). The reaction mixture was flushed with nitrogen, capped and heated to 80° C. for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, methyl (S)-1-((S)-2-(5-(4′-(3-((S)-1-(R)-2-(dimethylamino)-2-phenylacetyl)pyrrolidine-2-carbonyl)ureido)biphenyl-4-yl)-1H-imidazol-2-yl)pyrrolidin-1-yl)-3-methyl-1-oxobutan-2-ylcarbamate as a white solid, (126 mg, 31%): ESI-LRMS m/e calcd for C₄₂H₅₀N₈O₆ [M⁺] 762. found 763 [M+H⁺]; ¹H NMR (400 MHz, DMSO-d₆) δ ppm 0.77-0.94 (m, 6H) 1.22 (br. s., 1H) 1.75-2.06 (m, 7H) 2.12 (s, 7H) 3.42 (d, J=10.30 Hz, 1H) 3.52 (s, 3H) 3.73-3.86 (m, 2H) 4.04 (t, J=8.03 Hz, 1H) 4.17 (s, 2H) 4.35 (s, 1H) 5.06 (d, J=3.56 Hz, 2H) 7.21-7.79 (m, 14H) 10.47 (br. s., 1H) 10.86 (br. s., 1H) 11.76 (br. s., 1H).

### Example 5

### (S)-tert-Butyl 2-(2-(4-(2-((S)-1-((S)-2-(methoxycarbonylamino)-3-methylbutanoyl)pyrrolidin-2-yl)-1H-imidazol-5-yl)phenyl)benzo[d]thiazol-6-ylcarbamoylcarbamoyl)pyrrolidine-1-carboxylate

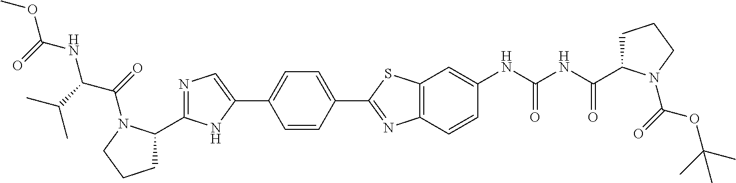

To a suspension of 2-bromo-6-nitrobenzo[d]thiazole (2.53 g, 9.75 mmol) in AcOH (48.7 mL) at room temperature was added iron powder (2.72 g, 48.7 mmol). The brown suspension was stirred at rt for 24 h. The reaction was diluted with H₂O (400 mL) and extracted with EtOAc (2×200 mL). The combined organic layers were washed with H₂O (200 mL) and sat. aq. NaCl (200 mL), dried over Na₂SO₄, filtered, and concentrated in vacuo. The crude material was purified by flash chromatography (80 g silica gel; 0-100% EtOAc-hexanes linear gradient) to provide 2-bromobenzo[d]thiazol-6-amine (1.25 g, 56%) as a brown solid. MS (ES+APCI⁺) m/z 229/231 [M+H]⁺; ¹H NMR (300 MHz, DMSO-d₆) δ 7.60 (d, J=8.7 Hz, 1H), 7.04 (d, J=2.6 Hz, 1H), 6.73-6.78 (m, 1H), 5.53 (s, 2H).

To a −78° C. solution of (S)-tert-butyl 2-carbamoylpyrrolidine-1-carboxylate (252 mg, 1.18 mmol) in THF (11.8 mL) was added phenyl chloroformate (163 μL, 1.29 mmol) followed by lithium bis(trimethylsilyl)amide (2.59 mL, 1 M in THF, 2.59 mmol) dropwise. The clear, orange solution was stirred at −78° C. for 2 h. The reaction was quenched by the addition of sat. aq. NH₄Cl (50 mL) and extracted with EtOAc (50 mL). The organic layer was washed with sat. aq. NaCl (50 mL), dried over Na₂SO₄, filtered, and concentrated in vacuo.

The crude material was taken up in MeCN (6.81 mL) and 2-bromobenzo[d]thiazol-6-amine (156 mg, 681 μmol) and N,N-diisopropylethylamine (236 μL, 1.36 mmol) were added. The reaction was stirred at rt for 20 h. The reaction was concentrated in vacuo and the crude material was purified by flash chromatography (40 g RediSep Gold silica gel; 0-100% EtOAc-hexanes linear gradient) to provide (S)-tert-butyl 2-(2-bromobenzo[d]thiazol-6-ylcarbamoylcarbamoyl)pyrrolidine-1-carboxylate (251 mg, 79%) as an off-white solid. [α]−19 (c 0.065, MeOH); MS (ES+APCI+) m/z 469/471 [M+H]+; ¹H NMR (300 MHz, DMSO-d6) δ 11.05-10.92 (m, 1H), 10.72, 10.63 (2×s, 1H), 8.44 (d, J=2.3 Hz, 1H), 7.93 (d, J=8.7 Hz, 1H), 7.58 (dd, J=8.7, 2.3 Hz, 1H), 4.36-4.25 (m, 1H), 3.46-3.29 (m, 2H), 2.29-2.11 (m, 1H), 1.99-1.75 (m, 3H), 1.40, 1.32 (2×s, 9H).

To a suspension of (S)-tert-butyl 2-(2-bromobenzo[d]thiazol-6-ylcarbamoylcarbamoyl)pyrrolidine-1-carboxylate (248 mg, 528 μmol) in DME at room temperature (4.23 mL) and H₂O (1.06 mL) was added methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (289 mg, 581 μmol) and sodium bicarbonate (133 mg, 1.59 mmol). The mixture was sparged with Ar for 30 min, then [1,1′-bis(diphenylphosphino)ferrocene]dichloropalladium(II)dichloromethane complex (43.2 mg, 52.8 μmol) was added. The reaction was sealed and stirred at 80° C. for 8 h. The reaction was cooled to rt and concentrated in vacuo. The residue was diluted with sat. aq. NaHCO₃ (50 mL) and extracted with CH₂Cl₂ (2×50 mL). The combined organic layers were washed with sat. aq. NaCl (50 mL), dried over Na₂SO₄, filtered, and concentrated in vacuo. The crude material was purified by flash chromatography (40 g RediSep Gold silica gel; 0-10% MeOH—CH₂Cl₂ linear gradient). The mixed fractions were re-purified (12 g RediSep Gold silica gel; 0-10% MeOH—CH₂Cl₂ linear gradient) and the clean fractions from both columns were combined to provide (S)-tert-butyl 2-(2-(4-(2-((S)-1-(S)-2-(methoxycarbonylamino)-3-methylbutanoyl)pyrrolidin-2-yl)-1H-imidazol-5-yl)phenyl)benzo[d]thiazol-6-ylcarbamoylcarbamoyl)pyrrolidine-1-carboxylate (228 mg, 57%) as a yellow solid. [α]−119 (c 0.090, MeOH); MS (ES+APCI)⁺ m/z 759 [M+H]⁺; ¹H NMR (300 MHz, DMSO-d₆) δ 12.42-11.88 (m, 1H), 11.04-10.89 (m, 1H), 10.76-10.59 (m, 1H), 8.46-8.42 (m, 1H), 8.10-7.56 (7H), 7.40-7.24 (m, 1H), 5.29-5.04 (m, 1 H), 4.39-4.26 (m, 1H), 4.11-3.75 (m, 3H), 3.54 (s, 3H), 3.47-3.33 (m, 2H), 2.25-1.71 (m, 9 H), 1.45-1.30 (m, 9H), 0.95-0.81 (m, 6H).

### Example 6

### [(S)-1-((S)-2-{5-[4-(6-[3-[(S)-1-((S)-2-Methoxycarbonylamino-3-methyl-butyryl)-pyrrolidine-2-carbonyl]-ureido}-benzothiazol-2-yl)-phenyl]-1H-imidazol-2-yl]-pyrrolidine-1-carbonyl)-2-methyl-propyl]-carbamic acid methyl ester

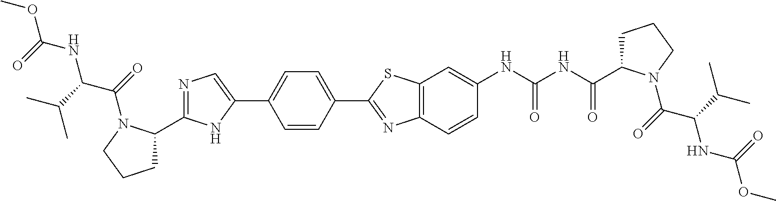

To a solution of (S)-tert-butyl 2-(2-(4-(2-((S)-1-(S)-2-(methoxycarbonylamino)-3-methylbutanoyl)pyrrolidin-2-yl)-1H-imidazol-5-yl)phenyl)benzo[d]thiazol-6-ylcarbamoylcarbamoyl)pyrrolidine-1-carboxylate (93.5 mg, 123 μmol) in CH₂Cl₂ (1.11 mL) at 23° C. was added TFA (123 μL). The clear, orange solution was stirred at rt for 24 h. The reaction was concentrated in vacuo. The residue was concentrated from PhMe (2×) then taken up in CH₂Cl₂-MeOH and treated with Si-carbonate (587 mg, 0.63 g/mmol loading, 370 μmol). The mixture was stirred at rt for 1 h then filtered, and the filtrate was concentrated in vacuo to provide methyl (S)-3-methyl-1-oxo-1-((S)-2-(5-(4-(6-(3-(S)-pyrrolidine-2-carbonylureido)benzo[d]thiazol-2-yl)phenyl)-1H-imidazol-2-yl)pyrrolidin-1-yl)butan-2-ylcarbamate (81 mg, quant.) as a light yellow solid. [α]−91 (c 0.050, MeOH); MS (ES+APCI⁺) m/z 659 [M+H]⁺.

To a solution of methyl (S)-3-methyl-1-oxo-1-((S)-2-(5-(4-(6-(3-(S)-pyrrolidine-2-carbonylureido)benzo[d]thiazol-2-yl)phenyl)-1H-imidazol-2-yl)pyrrolidin-1-yl)butan-2-ylcarbamate (57.8 mg, 87.7 μmol) in DMF (175 μL) at 23° C. was added (S)-2-(methoxycarbonylamino)-3-methylbutanoic acid (16.9 mg, 96.5 μmol), followed by N,N-diisopropylethylamine (45.5 μL, 263 μmol) and HATU (40.0 mg, 105 μmol). The clear, orange solution was stirred at rt for 1 h. The reaction was diluted with MeCN—H₂O and purified by preparative HPLC (5-95% MeCN—H₂O w/0.1% TFA). The product was neutralized by treatment with Si-carbonate to provide [(S)-1-((S)-2-{5-[4-(6-{3-[(S)-1-((S)-2-methoxycarbonylamino-3-methyl-butyryl)-pyrrolidine-2-carbonyl]-ureido}-benzothiazol-2-yl)-phenyl]-1H-imidazol-2-yl}-pyrrolidine-1-carbonyl)-2-methyl-propyl]-carbamic acid methyl ester (12.2 mg, 17%) as a light yellow solid. [α]−192 (c 0.040, MeOH); MS (ES+APCI⁺) m/z 816 [M+H]⁺; ¹H NMR (300 MHz, DMSO-d₆) δ 12.42-11.88 (m, 1H), 10.97 (s, 1H), 10.58 (s, 1H), 8.45-8.38 (m, 1H), 8.10-7.53 (m, 7H), 7.45-7.37 (m, 1H), 7.33-7.26 (m, 1H), 5.28-5.04 (m, 1H), 4.54-4.44 (m, 1H), 4.12-3.96 (m, 2H), 3.90-3.75 (m, 3H), 3.70-3.59 (m, 1H), 3.57-3.51 (m, 6H), 2.25-1.83 (m, 10H), 1.00-0.80 (m, 12H).

### Example 7

### Methyl (S)-1-((S)-2-(5-(4-(6-(3-((S)-1-((R)-2-(dimethylamino)-2-phenylacetyl)pyrrolidine-2-carbonyl)ureido)benzo[d]thiazol-2-yl)phenyl)-1H-imidazol-2-yl)pyrrolidin-1-yl)-3-methyl-1-oxobutan-2-ylcarbamate

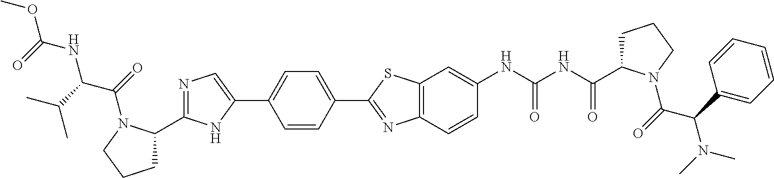

According to the procedure of Step 2-1, (S)-tert-butyl 2-(2-(4-(2-((S)-1-(S)-2-(methoxycarbonylamino)-3-methylbutanoyl)pyrrolidin-2-yl)-1H-imidazol-5-yl)phenyl)benzo[d]thiazol-6-ylcarbamoylcarbamoyl)pyrrolidine-1-carboxylate (107 mg, 141 mmol) was reacted to provide methyl (S)-3-methyl-1-oxo-1-((S)-2-(5-(4-(6-(3-(S)-pyrrolidine-2-carbonylureido)benzo[d]thiazol-2-yl)phenyl)-1H-imidazol-2-yl)pyrrolidin-1-yl)butan-2-ylcarbamate (93 mg, quant.).

To a rt solution of methyl (S)-3-methyl-1-oxo-1-((S)-2-(5-(4-(6-(3-(S)-pyrrolidine-2-carbonylureido)benzo[d]thiazol-2-yl)phenyl)-1H-imidazol-2-yl)pyrrolidin-1-yl)butan-2-ylcarbamate (90.7 mg, 138 μmol) in DMF (275 μL) was added (R)-2-(dimethylamino)-2-phenylacetic acid hydrochloride (32.7 mg, 151 μmol), followed by N,N-diisopropylethylamine (95.3 μL, 551 μmol) and HATU (62.8 mg, 165 μmol). The clear, orange solution was stirred at rt for 2 h. The reaction was diluted with MeCN—H₂O and purified by preparative HPLC (5-95% MeCN—H₂O w/0.1% TFA). The product was neutralized by treatment with Si-carbonate to provide methyl (S)-1-((S)-2-(5-(4-(6-(3-((S)-1-((R)-2-(dimethylamino)-2-phenylacetyl)pyrrolidine-2-carbonyl)ureido)benzo[d]thiazol-2-yl)phenyl)-1H-imidazol-2-yl)pyrrolidin-1-yl)-3-methyl-1-oxobutan-2-ylcarbamate (41.3 mg, 37%) as a light yellow solid. MS (ES+APCI⁺) m/z 820 [M+H]⁺; ¹H NMR (300 MHz, DMSO-d₆) δ 12.43-11.88 (m, 1H), 10.95 (s, 1H), 10.62 (s, 1H), 8.44-8.35 (m, 1H), 8.11-7.10 (m, 13H), 5.28-4.94 (m, 1H), 4.44-4.34 (m, 1H), 4.25-4.17 (m, 1H), 4.12-4.01 (m, 1H), 3.93-3.78 (m, 3H), 3.54 (s, 3H), 3.51-3.36 (m, 1H), 2.15 (s, 6H), 2.12-1.73 (m, 9H), 0.94-0.80 (m, 6H).

### Example 8

### ((S)-1-{(S)-2-[5-(4-(5-{3-[(S)-1-((R)-2-Methoxycarbonylamino-2-phenyl-acetyl)-pyrrolidine-2-carbonyl]-ureido}-pyridin-2-yl)-phenyl)-1H-imidazol-2-yl]-pyrrolidine-1-carbonyl}-2-methyl-propyl)-carbamic acid methyl ester

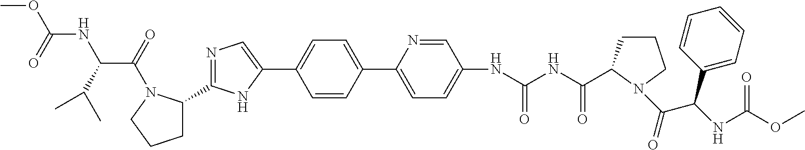

To an ice cooled mixture of 6-bromopyridin-3-amine (2.0 g, 11.60 mmol) in dioxane (20 mL) was added diphosgene (1.37 g, 6.94 mmol). A precipitate formed upon addition of the diphosgene and the resulting mixture was stirred at 0° C. for 2 h. Filter off the solids and wash with ether to afford 2-bromo-5-isocyanato-pyridine as a light red solid, (2.21 g, 96%), which was used without further purification: ESI-LRMS m/e calcd for C₆H₃BrN₂O [M⁺] 199.

A mixture of (S)-tert-butyl 2-carbamoylpyrrolidine-1-carboxylate (1.0 g, 4.67 mmol) and 2-bromo-5-isocyanato-pyridine (929 mg, 4.67 mmol) in toluene (10 mL) was stirred at reflux for 3 h. Upon completion of the reaction the solid was removed by filtration and the filtrate concentrated. The crude product obtained was purified by ISCO flash chromatography (Teledyne Isco RediSep Flash Column 40 g; (0% to 100% ethyl acetate/hexane) to afford, (S)-2-[3-(6-bromo-pyridin-3-yl)-ureidocarbonyl]-pyrrolidine-1-carboxylic acid tert-butyl ester as a white solid, (0.26 g, 14%): ESI-LRMS m/e calcd for C₁₆H₂₁BrN₄O₄ [M⁺] 413. found 414 [M+H⁺].

To a stirred mixture of (S)-2-[3-(6-bromo-pyridin-3-yl)-ureidocarbonyl]-pyrrolidine-1-carboxylic acid tert-butyl ester (250 mg, 0.61 mmol) dissolved in methanol (20 mL) was added a 4.0M HCl/dioxane solution (5 mL). After addition was complete the mixture was stirred at room temperature for 3 h. The reaction mixture was then concentrated in vacuo to afford, 1-(6-bromo-pyridin-3-yl)-3-(S)-pyrrolidine-2-carbonyl)-urea hydrochloride as a light yellow powder, (210 mg, 99%): ESI-LRMS m/e calcd for C₁₁H₁₃BrN₄O₂₋HCl [M⁺] 349.5. found 314 [M+H⁺] (free base).

N,N′-Diisopropylethylamine (233 mg, 1.80 mmol) was added to a solution of 1-(6-bromo-pyridin-3-yl)-3-(S)-pyrrolidine-2-carbonyl)-urea hydrochloride (210 mg, 0.60), (R)-2-(methoxycarbonylamino)-2-phenylacetic acid (BMS patent) (126 mg, 0.60 mmol) and HATU (228 mg, 0.60 mmol) in DMF (10 mL). After the addition was complete the reaction was stirred at room temperature for 4 h. The reaction was diluted with ethyl acetate and washed with water, 0.5N hydrochloric acid, a saturated sodium bicarbonate solution, a saturated sodium chloride solution and dried over magnesium sulfate, filtered and concentrated to afford, ((R)-2-{(S)-2-[3-(6-Bromo-pyridin-3-yl)-ureidocarbonyl]-pyrrolidin-1-yl}-2-oxo-1-phenyl-ethyl)-carbamic acid methyl ester as a yellow solid, (284 mg, 94%): ESI-LRMS m/e calcd for C₂₁H₂₂BrN₅O₅ [M⁺] 504. found 505 [M+H⁺].

In a sealed tube [1,1′-bis(diphenylphosphino)ferrocene]dichloropalladium (II) (40 mg, 0.05 mmol) was added to a mixture of methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (270 mg, 0.54 mmol) (BMS patent), ((R)-2-{(S)-2-[3-(6-Bromo-pyridin-3-yl)-ureidocarbonyl]-pyrrolidin-1-yl}-2-oxo-1-phenyl-ethyl)-carbamic acid methyl ester (274 mg, 0.0.54 mmol) and sodium bicarbonate (137 mg, 1.63 mmol) in 1,2-dimethoxyethane (6 mL) and water (1 mL). The reaction mixture was flushed with nitrogen, capped and heated to 80° C. for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, ((S)-1-{(S)-2-[5-(4-{5-{3-[(S)-1-(R)-2-Methoxycarbonylamino-2-phenyl-acetyl)-pyrrolidine-2-carbonyl]-ureido}-pyridin-2-yl)-1H-imidazol-2-yl}-pyrrolidine-1-carbonyl}-2-methyl-propyl)-carbamic acid methyl ester as a white solid, (12 mg, 3%): ESI-LRMS m/e calcd for C₄₁H₄₇N₉O₈ [M⁺] 793. found 794 [M+H⁺]; ¹H NMR (400 MHz, DMSO-d₆) δ ppm 0.79-0.93 (m, 6H) 1.24 (br. s., 2H) 1.77-2.10 (m, 5H) 2.17 (br. s., 2H) 2.33 (br. s., 1 H) 2.67 (br. s., 1H) 3.15 (br. s., 1H) 3.42 (br. s., 1H) 3.54 (br. s., 6H) 3.71 (br. s., 1H) 3.81 (br. s., 3H) 4.07 (br. s., 1H) 4.43 (br. s., 1H) 5.09 (br. s., 1H) 5.53 (d, J=8.53 Hz, 1H) 7.25-7.42 (m, 5H) 7.69-7.84 (m, 3H) 7.96 (d, J=6.53 Hz, 2H) 8.13 (br. s., 1H) 8.77 (br. s., 1H) 10.53 (br. s., 1H) 10.99 (br. s., 1H).

### Example 9

### {(4S,7S)-4-[5-(4′-{3-[(S)-1-[((R)-2-Methoxycarbonylamino-2-phenyl-acetyl)-pyrrolidine-2-carbonyl]-ureido}-biphenyl-4-yl)-1H-imidazol-2-yl}-6,10-dioxo-octahydro-pyridazino[1,2-a]diazepin-7-yl]-carbamic acid methyl ester

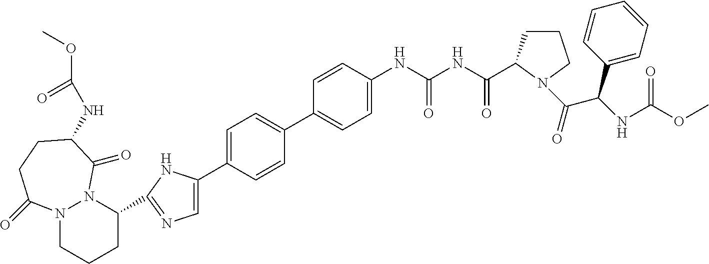

To a solution of (1S,9S)-tert-butyl 9-(1,3-dioxoisoindolin-2-yl)-6,10-dioxooctahydro-1H-pyridazino[1,2-a][1,2]diazepine-1-carboxylate (2.00 g, 4.68 mmol) in ethanol (10 mL) was added hydrazine (180 mg, 5.61 mmol). The reaction was stirred at room temperature for 3 h. The ethanol and excess hydrazine were concentrated in vacuo and the residue co-evaporated with ethanol to afford, (1S,9S)-tert-butyl 9-amino-6,10-dioxooctahydro-1H-pyridazino[1,2-a][1,2]diazepine-1-carboxylate as a white powder, (1.63 g, 100%): ESI-LRMS m/e calcd for C₁₄H₂₃N₃O₄ [M⁺] 297. found 298 [M+H⁺].

To an ice-cooled solution of (1S,9S)-tert-butyl 9-amino-6,10-dioxooctahydro-1H-pyridazino[1,2-a][1,2]diazepine-1-carboxylate (1.50 g, 5.04 mmol) in DMF (15 mL) was added sodium carbonate (642 mg, 6.05 mmol) followed by methyl chloroformate (524 mg, 5.55 mmol). After the addition was complete the ice bath was removed and the reaction stirred at room temperature for 2 h. The reaction mixture was diluted with ethyl acetate and washed with water 2N hydrochloric acid, a saturated sodium chloride solution and dried over magnesium sulfate, filtered and concentrated to afford, (1S,9S)-tert-butyl 9-(methoxycarbonylamino)-6,10-dioxooctahydro-1H-pyridazino[1,2-a][1,2]diazepine-1-carboxylate as a white solid, (1.28 g, 71%): ESI-LRMS m/e calcd for C₁₆H₂₅N₃O₆ [M⁺] 355. found 356 [M+H⁺].

To a solution of (1S,9S)-tert-butyl 9-(methoxycarbonylamino)-6,10-dioxooctahydro-1H-pyridazino[1,2-a][1,2]diazepine-1-carboxylate (1.25 g, 3.52 mmol) dissolved into methylene chloride (10 mL) was added trifluoroacetic acid (10 mL). The reaction was stirred at room temperature for 1 h and concentrated in vacuo. Toluene (5 mL) was added and the reaction concentrated in vacuo to afford (1S,9S)-9-(methoxycarbonylamino)-6,10-dioxooctahydro-1H-pyridazino[1,2-a][1,2]diazepine-1-carboxylic acid as a white solid, (537 mg, 51%): ESI-LRMS m/e calcd for C₁₂H₇N₃O₆ [M⁺] 299. found 300 [M+H⁺].

N,N′ diisopropylethylamine (680 mg, 5.26 mmol) was added dropwise at room temperature to a heterogeneous mixture of (1S,9S)-9-(methoxycarbonylamino)-6,10-dioxooctahydro-1H-pyridazino[1,2-a][1,2]diazepine-1-carboxylic acid (525 mg, 1.75 mmol), 2-amino-1-(4-bromo-phenyl)-ethanone hydrochloride (439 mg, 1.75 mmol), HATU (667 mg, 1.75 mmol) and DMF (10 mL). After addition was complete the reaction was stirred at room temperature for 4 h. The reaction mixture was diluted with ethyl acetate and washed with water, 1N hydrochloric acid, a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (30% to 100% ethyl acetate/hexane) to afford, methyl (4S,7S)-4-(2-(4-bromophenyl)-2-oxoethylcarbamoyl)-6,10-dioxooctahydro-1H-pyridazino[1,2-a][1,2]diazepin-7-ylcarbamate as a light yellow solid, (587 mg, 68%): ESI-LRMS m/e calcd for C₂₀H₂₃BrN₄O₆ [M⁺] 495. found 496 [M+H⁺].

A mixture of methyl (4S,7S)-4-(2-(4-bromophenyl)-2-oxoethylcarbamoyl)-6,10-dioxooctahydro-1H-pyridazino[1,2-a][1,2]diazepin-7-ylcarbamate (500 mg, 1.01 mmol) and ammonium acetate (389 mg, 5.05 mmol) in xylenes (10 mL) was heated in a sealed tube at 140° C. for 4 h. The reaction was then cooled to room temperature and diluted with ethyl acetate. The organic fraction was washed with a saturated sodium bicarbonate solution, a saturated sodium chloride solution and dried over magnesium sulfate, filtered and concentrated. The crude product obtained was purified by ISCO flash chromatography (Teledyne Isco RediSep Flash Column 40 g; (30% to 100% ethyl acetate/hexane) to afford, methyl (4S,7S)-4-(5-(4-bromophenyl)-1H-imidazol-2-yl)-6,10-dioxooctahydro-1H-pyridazino[1,2-a][1,2]diazepin-7-ylcarbamate as a yellow solid, (435 mg, 91%): ESI-LRMS m/e calcd for C₂₀H₂₂BrN₅O₄ [M⁺] 476. found 477 [M+H⁺].

1,1′-bis(diphenylphosphino)ferrocene-palladium(II)dichloride dichloromethane complex (159 mg, 0.20 mmol) was added to a sealed tube containing a mixture of methyl (4S,7S)-4-(5-(4-bromophenyl)-1H-imidazol-2-yl)-6,10-dioxooctahydro-1H-pyridazino[1,2-a][1,2]diazepin-7-ylcarbamate (925 mg, 1.94 mmol), 4,4,4′,4′,5,5,5′,5′-octamethyl-2,2′-bi(1,3,2-dioxaborolane) (1.48 g, 5.83 mmol), potassium acetate (953 mg, 9.71 mmol) and 1,4-dioxane (40 ml). The vessel was purged with nitrogen, capped and heated with an oil bath at 80° C. overnight. Cool the reaction to room temperature and filter through celite. Concentrate the reaction in vacuo. The crude mixture was diluted with methylene chloride and washed with water, a saturated sodium chloride solution and dried over magnesium sulfate, filtered and concentrated. The crude product obtained was purified by ISCO flash chromatography (Teledyne Isco RediSep Flash Column 40 g; (50% to 100% ethyl acetate/hexane) to afford, methyl (4S,7S)-6,10-dioxo-4-(5-(4-(4,4,5,5-tetramethyl-1,3,2-dioxaborolan-2-yl)phenyl)-1H-imidazol-2-yl)octahydro-1H-pyridazino[1,2-a][1,2]diazepin-7-ylcarbamate as a brown solid, (358 mg, 35%): ESI-LRMS m/e calcd for C₂₆H₃₄BN₅O₆ [M⁺] 523. found 524 [M+H⁺].

In a sealed tube [1,1′-bis(diphenylphosphino)ferrocene]dichloropalladium (II) (10 mg, 0.02 mmol) was added to a mixture of methyl (4S,7S)-6,10-dioxo-4-(5-(4-(4,4,5,5-tetramethyl-1,3,2-dioxaborolan-2-yl)phenyl)-1H-imidazol-2-yl)octahydro-1H-pyridazino[1,2-a][1,2]diazepin-7-ylcarbamate (70 mg, 0.13 mmol), ((R)-2-{(S)-2-[3-(4-Bromo-phenyl)-ureidocarbonyl]-pyrrolidin-1-yl}-2-oxo-1-phenyl-ethyl)-carbamic acid methyl ester (67 mg, 0.13 mmol) and sodium bicarbonate (34 mg, 0.0.40 mmol) in 1,2-dimethoxyethane (6 mL) and water (1 mL). The reaction mixture was flushed with nitrogen, capped and heated to 80° C. for 16 h. The reaction mixture was concentrated and partitioned between 20% methanol/methylene chloride and water and the aqueous phase extracted with 20% methanol/methylene chloride. The combined organic phases were washed with a saturated sodium chloride solution and dried over magnesium sulfate, filtered and concentrated. The crude product obtained was purified by reverse phase HPLC using a 50 g Polaris C18A column eluting with acetonitrile/water (30% to 100%) to afford, {(4S,7S)-4-[5-(4′-{3-[(S)-1-(R)-2-Methoxycarbonylamino-2-phenyl-acetyl)-pyrrolidine-2-carbonyl]-ureido}-biphenyl-4-yl)-1H-imidazol-2-yl}-6,10-dioxo-octahydro-pyridazino[1,2-a]diazepin-7-yl]-carbamic acid methyl ester as a white solid, (4 mg, 4%): ESI-LRMS m/e calcd for C₄₂H₄₅N₉O₉ [M⁺] 819. found 820 [M+H⁺]; 1; ¹H NMR (400 MHz, DMSO-d₆) δ ppm 1.54-1.64 (m, 1H) 1.69-1.95 (m, 6H) 2.12-2.22 (m, 1H) 2.26-2.35 (m, 1H) 2.37-2.48 (m, 1H) 2.55-2.76 (m, 2H) 3.14-3.21 (m, 1H) 3.32-3.38 (m, 1H) 3.57 (s, 3H) 3.64 (s, 3H) 3.65-3.70 (m, 1H) 4.35-4.40 (m, 1H) 5.52-4.70 (m, 2H) 5.24-5.27 (dd, 1H) 5.51-5.59 (m, 1H) 6.17 (d, 1H) 7.21-7.27 (m, 5H) 7.55-7.80 (m, 10H) 9.00 (s, 1H) 9.90 (s, 1H) 11.70 (br. s., 1H).

### Example 10

### ((S)-1-{(S)-2-[5-(4′-{3-[(S)-1-[((S)-2-Methoxycarbonylamino-3-methyl-butyryl)-pyrrolidine-2-carbonyl]-ureidomethyl}-biphenyl-4-yl)-1H-imidazol-2-yl]-pyrrolidine-1-carbonyl}-2-methyl-propyl)-carbamic acid methyl ester

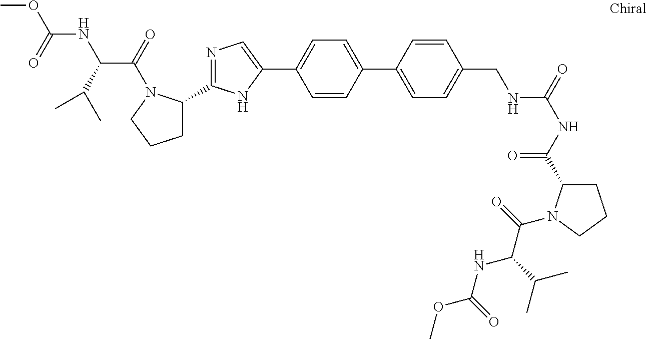

A suspension of 1-bromo-4-(isocyanatomethyl)benzene (920 mg, 4.34 mmol), and (S)-tert-butyl 2-carbamoylpyrrolidine-1-carboxylate (930 mg, 4.34 mmol) was in toluene (10 ml) was heated at 120° C. for 4 h under nitrogen. The reaction mixture was then cooled to 23° C. and let stand for 16 h. This resulted in the formation of crystalline solid, which was removed by filtration. The filtrate was then concentrated and purified on ISCO flash chromatography (Teledyne Isco RediSep Flash Column 12 g; (0% to 100% ethyl acetate/hexane) to afford (S)-2-[3-(4-Bromo-benzyl)-ureidocarbonyl]-pyrrolidine-1-carboxylic acid tert-butyl ester as a white foam (503.4 mg, 26.7% yield): ESI-LRMS m/e calcd for C₁₈H₂₄BrN₃O₄ [M⁺] 426. found 428 [M+H⁺].

To a mixture of methyl (S)-3-methyl-1-oxo-1-((S)-2-(5-(4-(4,4,5,5-tetramethyl-1,3,2-dioxaborolan-2-yl)phenyl)-1H-imidazol-2-yl)pyrrolidin-1-yl)butan-2-ylcarbamate (571 mg, 1.15 mmol, Eq: 1.00), tert-butyl 2-(4 bromobenzylcarbamoyl-carbamoyl)pyrrolidine-1-carboxylate (500 mg, 1.15 mmol, Eq: 1.00) and sodium bicarbonate (290 mg, 3.45 mmol, Eq: 3.00) in 1,2-dimethoxyethane (6 ml) and Water (1 ml) was added [1,1′-bis(diphenylphosphino)ferocene]-dichloropalladium(II) (84.1 mg, 115 μmol, Eq: 0.10) was added. The reaction mixture was flushed with nitrogen, capped and heated in an oil bath (80° C.) for 16 hrs. The reaction mixture was concentrated under vacuum. The aqueous work up using 20% methanol/methylene chloride followed by purification by reverse phase HPLC using a 50 g Polaris C18A column eluting with CH₃CN/water (30% to 100%) gradient to afford 243-(4′-}2-[(S)-1-((S)-2-methoxycarbonylamino-3-methyl-butyryl)-pyrrolidin-2-yl]-3H-imidazol-4-yl}-biphenyl-4-ylmethyl)-ureidocarbonyl]-pyrrolidine-1-carboxylic acid tert-butyl ester (226.3 mg, yield 23.4%) as a light brown powder. ESI-LRMS m/e calcd for C₃₈H₄₉BrN₇O₇ [M⁺]715 found 716 [M+H⁺].

A solution of tert-butyl 2-((4′-(2-((S)-1-((S)-2-(methoxycarbonylamino)-3-methylbutanoyl)pyrrolidin-2-yl)-1H-imidazol-5-yl)biphenyl-4-yl)methylcarbamoylcarbamoyl)pyrrolidine-1-carboxylate (220 mg, 307 μmol, Eq: 1.00) in CH₃OH (15 ml) was treated with a solution of 4N HCl/dioxane (15.0 ml). The reaction mixture was stirred for 4.5 h at 25° C. The solution was then, concentrated to afford a 2:1 mixture of desired {(S)-2-Methyl-1-[(S)-2-(5-{4′-[3-(pyrrolidine-2-carbonyl)-ureidomethyl]-biphenyl-4-yl}-1H-imidazol-2-yl)-pyrrolidine-1-carbonyl]-propyl}-carbamic acid methyl ester dihydrochloride contaminated with ((S)-2-Methyl-1-{(S)-2-[5-(4′-ureidomethyl-biphenyl-4-yl)-1H-imidazol-2-yl]-pyrrolidine-1-carbonyl}-propyl)-carbamic acid methyl ester as light brown solid (237.8 mg, yield 74%). ESI-LRMS m/e calcd for C₃₃H₄₁N₇O₅ [M⁺]615 found 616 [M+H⁺].

A suspension of methyl (2S)-3-methyl-1-oxo-1-((2S)-2-(5-(4′-((3-pyrrolidine-2-carbonylureido)methyl)biphenyl-4-yl)-1H-imidazol-2-yl)pyrrolidin-1-yl)butan-2-ylcarbamate dihydrochloride (118.9 mg, 114 μmol) in DMF (5 mL) was combined with (S)-2-(methoxycarbonylamino)-3-methylbutanoic acid (20.6 mg, 114 μmol, Eq: 1.00), HATU (50.9 mg, 134 μmol, Eq: 2.05) in DMF (2.5 ml). The mixture was then treated with N,N′-diisopropylethylamine (59.5 μl, 342 μmol). The reaction mixture was stirred for 16 h at 23° C. The reaction mixture, after aqueous work up using ethyl acetate/water followed by purification by reverse phase HPLC using a 50 g Polaris C18A column eluting with CH₃CN/water (30% to 100%) gradient to afford ((S)-1-{(S)-2-[5-(4′-{3-[(S)-1-(S)-2-Methoxycarbonylamino-3-methyl-butyryl)-pyrrolidine-2-carbonyl]-ureidomethyl}-biphenyl-4-yl)-1H-imidazol-2-yl]-pyrrolidine-1-carbonyl}-2-methyl-propyl)-carbamic acid methyl ester as an off-white powder (15.9 mg, yield —18.1%). ESI-LRMS m/e calcd for C₄₀H₅₂N₈O₈ [M⁺]772 found 773 [M+H⁺]. ¹H NMR (500 MHz, DMSO-d6) δ 11.51 (br. s., 1H), 10.20 (br. s., 1H), 8.50 (br. s., 1H), 7.77 (d, J=7.81 Hz, 2H), 7.53-7.70 (m, 5H), 7.29-7.45 (m, 3H), 5.11 (br. s., 1H), 4.31-4.53 (m, 3H), 3.97-4.17 (m, 2H), 3.77 (br. s., 3H), 3.48-3.65 (m, 7H), 2.14 (d, J=5.86 Hz, 3H), 1.77-2.06 (m, 7H), 0.74-1.01 (m, 12H).

### Example 11

### ((S)-1-{(S)-2-[5-(4′-{3-[(S)-1-((R)-2-Methoxycarbonylamino-2-phenyl-acetyl)-pyrrolidine-2-carbonyl]-ureidomethyl}-biphenyl-4-yl)-1H-imidazol-2-yl]-pyrrolidine-1-carbonyl}-2-methyl-propyl)-carbamic acid methyl ester

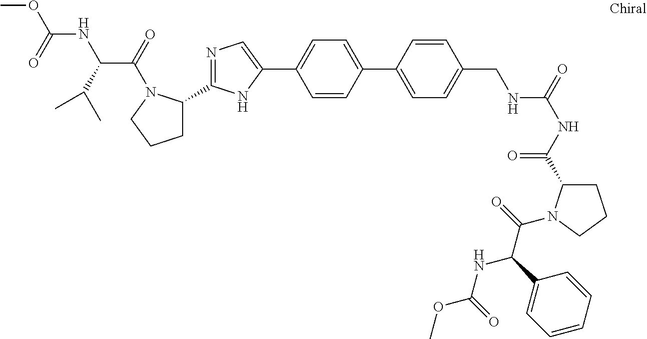

A suspension of methyl (2S)-3-methyl-1-oxo-1-((2S)-2-(5-(4′-((3-pyrrolidine-2-carbonylureido)methyl)biphenyl-4-yl)-1H-imidazol-2-yl)pyrrolidin-1-yl)butan-2-ylcarbamate dihydrochloride (118.9 mg, 114 μmol) (prepared in the example 10), in DMF (5 ml) to give a light brown suspension and (S)-2-(methoxycarbonylamino)-2-phenylacetic acid (23.8 mg, 114 mmol), HATU (44.2 mg, 114 μmol, Eq: 1.00) in DMF (2.5 ml) were added. Then, the mixture was treated with N,N′-diisopropylethylamine (59.5 μl, 342 μmol). The reaction mixture was stirred for 16 h at 23° C. The reaction mixture, after aqueous work up using ethyl acetate/water followed by purification by reverse phase HPLC using a 50 g Polaris C18A column eluting with CH₃CN/water (30% to 100%) gradient to afford ((S)-1-{(S)-2-[5-(4′-{3-[(S)-1-((R)-2-Methoxycarbonylamino-2-phenyl-acetyl)-pyrrolidine-2-carbonyl]-ureidomethyl}-biphenyl-4-yl)-1H-imidazol-2-yl]-pyrrolidine-1-carbonyl}-2-methyl-propyl)-carbamic acid methyl ester as off-white powder. (15.9 mg, yield—16.4%). ESI-LRMS m/e calcd for C₄₃H₅₀N₈O₈ [M⁺] 806. found 807 [M+H⁺]. ¹H NMR (500 MHz, DMSO-d6) δ 11.51 (br. s., 1H), 10.18 (br. s., 1H), 8.55 (br. s., 1H), 7.78 (d, J=7.81 Hz, 2H), 7.55-7.71 (m, 4H), 7.22-7.54 (m, 7H), 5.67 (d, J=0.98 Hz, 1H), 5.46 (d, J=7.81 Hz, 1H), 5.11 (br. s., 1H), 4.42 (br. s., 2H), 4.06-4.16 (m, 1H), 3.66-3.86 (m, 2H), 3.48-3.59 (m, 6H), 3.18 (d, J=8.30 Hz, 1H), 1.66-2.28 (m, 8H), 0.77-0.95 (m, 6H).

### Example 12

### ((S)-1-{(S)-2-[5-(3′-Fluoro-4′-{3-[(S)-1-[((S)-2-methoxycarbonylamino-3-methyl-butyryl)-pyrrolidine-2-carbonyl]-ureido}-biphenyl-4-yl)-1H-imidazol-2-yl]-pyrrolidine-1-carbonyl}-2-methyl-propyl)-carbamic acid methyl ester

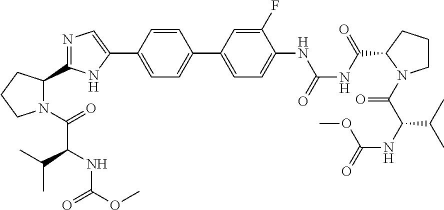

(S)-tert-Butyl 2-carbamoylpyrrolidine-1-carboxylate (674 mg, 3.14 mmol) was mixed with 4-bromo-2-fluoro-1-isocyanatobenzene (679 mg, 3.14 mmol) in toluene (12 mL) inside a sealed tube. The mixture was stirred under an oil bath heated to 135° C. for 4 hrs. Solvents were evaporated and the residue was purified through flash column chromatography (40 g silica gel, 0% to 40% ethyl acetate in hexanes in 25 minutes) to give (S)-2-[3-(4-bromo-2-fluoro-phenyl)-ureidocarbonyl]-pyrrolidine-1-carboxylic acid tert-butyl ester as a white waxy material (1.08 g, 80% yield). LC/MS calcd for C₁₇H₂₁BrFN₃O₄ 429.07 (M⁺). found 428.0 (M⁺−H).

(S)-tert-Butyl 2-(4-bromo-2-fluorophenylcarbamoylcarbamoyl)pyrrolidine-1-carboxylate (1.05 g, 2.44 mmol) was dissolved in 10 mL of ethyl acetate and hydrogen chloride in ether (5N, 5 mL) was added. The mixture was stirred at room temperature overnight. TLC indicated no more starting material. The mixture was treated with dry ether (20 mL) and solvents were decanted. The white solid was dried in vacuum to give a pure desired compound as 1-(4-bromo-2-fluoro-phenyl)-3-(S)-pyrrolidine-2-carbonyl)-urea (0.87 g, 97.2% yield). LC/MS calcd for C₁₂H₁₃BrFN₃O₂ 329.02 (M⁺). found 329.9 (M⁺+H).

(S)—N-(4-Bromo-2-fluorophenylcarbamoyl)pyrrolidine-2-carboxamide hydrochloride (0.84 g, 2.29 mmol), (S)-2-(methoxycarbonylamino)-3-methylbutanoic acid (442 mg, 2.52 mmol, prepared according to the procedure described in US2009/0202478) and HATU (958 mg, 2.52 mmol) were suspended in 25 mL of dry DMF and cooled under ice bath. Triethylamine (1 mL, 7.2 mmol) was added and the solution was stirred for 1 hr. The mixture was gradually warmed to room temperature and stirred overnight. Solvents were evaporated and the residue was extracted with ethyl acetate and 0.2N hydrochloric acid. The organic layer was washed with 0.1N hydrochloric acid, water and concentrated sodium bicarbonate solution, dried and evaporated to dryness. The residue was triturated with hexanes to give a white solid as ((S)-1-{(S)-2-[3-(4-bromo-2-fluoro-phenyl)-ureidocarbonyl]-pyrrolidine-1-carbonyl}-2-methyl-propyl)-carbamic acid methyl ester (1.12 g, 100% yield). LC/MS calculated for C₁₉H₂₄BrFN₄O₅ 486.09 (M⁺). found 485.0 (M⁺−H).

((S)-1-{(S)-2-[3-(4-Bromo-2-fluoro-phenyl)-ureidocarbonyl]-pyrrolidine-1-carbonyl}-2-methyl-propyl)-carbamic acid methyl ester (243 mg, 0.499 mmol), methyl (S)-1-(ethyl((R)-1-(5-(4-(4,4,5,5-tetramethyl-1,3,2-dioxaborolan-2-yl)phenyl)-1H-imidazol-2-yl)propyl)amino)-3-methyl-1-oxobutan-2-ylcarbamate (307 mg, 0.599 mmol), [1,1′-bis(diphenylphosphino)ferrocene]dichloropalladium (II) (36.5 mg, 0.0499 mmol), and sodium bicarbonate (126 mg, 1.5 mmol) were combined in 6 mL of DME and 1 mL of de-ionized water. The mixture was degassed with argon for 10 minutes and sealed. The mixture was stirred at 85° C. for 5 hrs and solvents were evaporated. The residue was extracted with ethyl acetate and 0.1N hydrochloride solution. The organic layer was washed with water, dried and concentrated. The residue was purified through reverse phase HPLC to give a light brown lyophilized powder (34 mg, 8.8% yield). LC/MS calcd for C₃₉H₄₉FN₈O₈ calcd 776.37 (M⁺). found 777.3 (M⁺+H); +). ¹H NMR (400 MHz, DMSO-d6) δ ppm 0.79 (d, J=6.8 Hz, 3H), 0.83 (d, J=6.8 Hz, 3H), 0.90 (d, J=6.6 Hz, 3H), 0.94 (d, J=6.6 Hz, 3H), 1.85-2.43 (m, 10H), 3.53 (s, 3H), 3.54 (s, 3H), 3.59-3.92 (m, 4H), 4.03 (t, J=8.3 Hz, 1H), 4.11 (t, J=8.0 Hz, 1H), 4.48 (m, 1H), 5.12 (t, J=6.9 Hz, 1H), 7.34 (d, J=8.4 Hz, 1H), 7.40 (d, J=8.3 Hz, 1H), 7.65 (d, J=8.6 Hz, 1H), 7.78 (dd, J=12.6, 1.8 Hz, 1H), 7.84 (d, J=8.3 Hz, 2H), 7.91 (d, J=8.4 Hz, 2H), 8.07-8.15 (m, 1H), 8.26 (t, J=8.6 Hz, 1H), 10.79 (s, 1H), 11.13 (s, 1H), 14.52 (br. s., 1H).

### Biological Examples

Determination of compounds HCV GT1b inhibitory replicon activity using the replicon luciferase reporter assay

The 2209-23 cell line was developed at Roche by stable transfection of the hepatoma cell line Huh-7 with a GT-1b Con1 subgenomic bicistronic replicon as previously described. Subgenomic replicon cell line was established in cured Huh7 cells, obtained from R. Bartenschlager (J. Virol. 2003 March; 77 (5):3007-19) The GT-1a H77 subgenomic replicon vector pRLuc H771b 75 S/I, was created by replacing the non structural region of the GT-1b Con1 subgenomic replicon by the one of the H77 strain, except for the first 75 amino acids of the NS3 protein that are from GT-1b Con1 strain. (J. Virol. 2001 77:5352-59) The GT-1a pRLuc H771b 75 S/I subgenomic replicon cell line was established in cured Huh7 cells, obtained from R. Bartenschlager. (J. Virol. 2003 March; 77 (5):3007-19)

All the subgenomic replicon cell lines were cultured in Dulbecco's Modified Eagle Medium (DMEM-Glutamax™-I; Invitrogen Cat #10569-010). The medium was supplemented with 10% Fetal Bovine Serum (Invitrogen Cat #10082-147), 1% penicillin/streptomycin (Mediatech Cat #30-002-CI) and 500 μg/ml of G418 (Mediatech Cat #30-234-CI). Cells were maintained at 37° C. in a humidified 5% CO₂ atmosphere.

2209-23 cells were plated at a cell density of 5000 cells per well in 96 well plates (Becton Dickinson, Cat #35 3296). Cells were plated in 90 μl of Dulbecco's Modified Eagle Medium (DMEM-Glutamax™-I), (Invitrogen Cat #10569-010) medium was supplemented with 5% Fetal Bovine Serum (Invitrogen Cat #10082-147), 1% penicillin/streptomycin (Mediatech Cat #30-002-CI). The pRluc H771b 75 S/I cells were plated in 96-well plate at 3000 cells/well in DMEM-Glutamax™-I containing 5% FBS and 1% penicillin/streptomycin in 90 μl final volume. Cells were allowed to equilibrate for 24 hours at 37° C. and 5% CO2 at which time compounds were added. Compounds (or medium as a control) were added 24 hours post-plating in 3 fold dilutions at a final DMSO concentration of 1% in 10 ul volume. Renilla luciferase reporter signal was read 72 hours after addition of compounds using the Renilla Luciferase Assay System (Promega, cat #E2820). EC50 values were defined as the compound concentration at which a 50% reduction in the levels of renilla luciferase reporter was observed as compared to control samples in the absence of compound and was determined by non-linear fitting of compound dose-response data. The EC50 was approximated if maximum percentage inhibition was less than 90% and more than 70%.

Determination of compounds cytotoxicity using the HCV GT1b replicon cell line measuring WST1.

2209-23 cells were plated at a cell density of 5000 cells per well in clear flat-bottom 96 well plate (Becton Dickinson, Cat #35 3075) for cell viability studies. The WST-1 cell proliferation assay (Roche Diagnostic, Cat#11644807001) was used to determine cell viability. Assay plates were set up in the same format as in the replicon assay. After 3 days of compound incubation 10 μl of WST-1 reagent was added to each well for 2 hours at 37° C. and 5% CO₂, following manufacturer's instructions. Absorption reading at 450 nm (reference filter at 650 nm) was determined using MRX Revelation microtiter plate reader (Lab System). CC₅₀ values were defined as the compound concentration required for reducing cell viability by 50% as compared to the untreated control in absence of compound and was determined by non-linear fitting of compound dose-response data. Representative assay data can be found in Table II below:

TABLE II

| | GT-1b IC₅₀ |
|---|---|
| Compound # | (nM) |
| I-1 | 0.463 |
| I-2 | 8.423 |
| I-3 | 0.009 |
| I-4 | 0.007 |
| I-5 | 1.984 |
| I-6 | 6.742 |
| I-7 | 0.427 |
| I-8 | 7.749 |
| I-9 | 0.344 |
| I-10 | 0.041 |
| I-11 | 0.029 |
| I-12 | 0.064 |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound of formula I 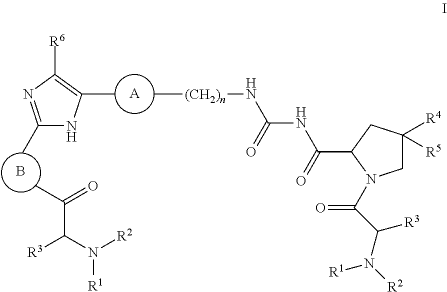
wherein:
R¹ and R² are independently H, lower alkyl, COOR⁷, COR^{S}, or CONHR⁹;
R³ is lower alkyl or aryl;
R⁴ and R⁵ are independently H or halo;
R⁶ is H or halo;
n is 0 or 1;
R⁷, R⁸, and R⁹ are independently H or lower alkyl;
A is 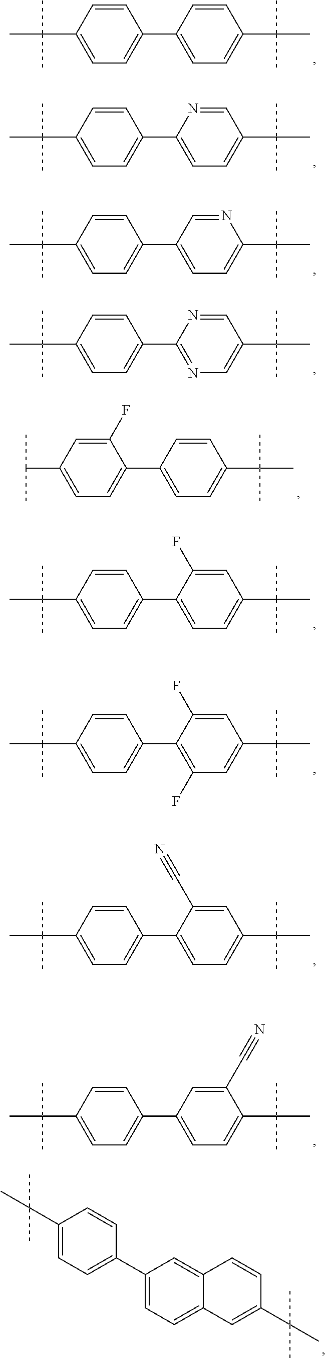 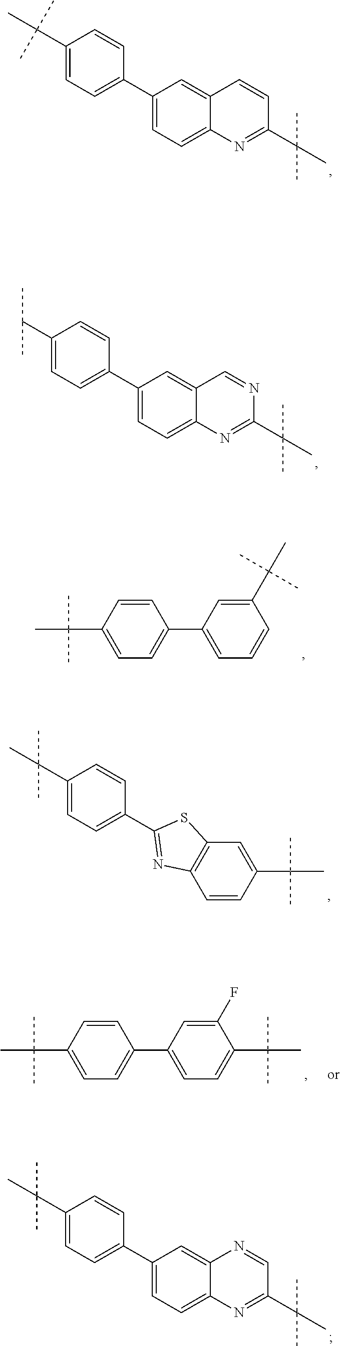
and
B is 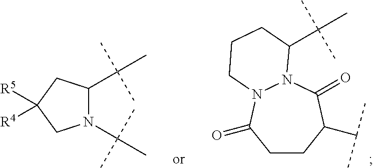
or a pharmaceutically acceptable salt thereof.

2. The compound of claim 1, wherein B is

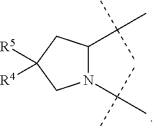

3. The compound of claim 2, wherein both R¹ are H, both R² are COOCH₃, and R⁶ is H.

4. The compound of claim 3, wherein n is 0.

5. The compound of claim 4, wherein A is biphenyl.

6. The compound of claim 4, wherein A is 2-Phenyl-benzothiazole.

7. The compound of claim 5, wherein both R³ are isopropyl.

8. The compound of claim 5, wherein one R³ is isopropyl and the other is phenyl.

9. The compound of claim 5, wherein both R⁴ are H and both R⁵ are H.

10. The compound of claim 5, wherein both R⁴ are F and both R⁵ are F.

11. A compound selected from the group consisting of:
((S)-1-{(S)-2-[3-(4′-{2-[(S)-1-(S)-2-Methoxycarbonylamino-3-methyl-butyryl)-pyrrolidin-2-yl]-3H-imidazol-4-yl}-biphenyl-4-yl)-ureidocarbonyl]-pyrrolidine-1-carbonyl}-2-methyl-propyl)-carbamic acid methyl ester;
((S)-1-{(S)-2-[3-(4′-{2-[(S)-1-(S)-2-Methoxycarbonylamino-3-methyl-butyryl)-pyrrolidin-2-yl]-3H-imidazol-4-yl}-biphenyl-3-yl)-ureidocarbonyl]-pyrrolidine-1-carbonyl}-2-methyl-propyl)-carbamic acid methyl ester;
((S)-1-{(S)-2-[5-(4′-{3-[(S)-1-(R)-2-Methoxycarbonylamino-2-phenyl-acetyl)-pyrrolidine-2-carbonyl]-ureido}-biphenyl-4-yl)-1H-imidazol-2-yl]-pyrrolidine-1-carbonyl}-2-methyl-propyl)-carbamic acid methyl ester;
((S)-1-{(S)-2-[5-(4′-{3-[(S)-1-(R)-2-Dimethylamino-2-phenyl-acetyl)-pyrrolidine-2-carbonyl]-ureido}-biphenyl-4-yl)-1H-imidazol-2-yl]-pyrrolidine-1-carbonyl}-2-methyl-propyl)-carbamic acid methyl ester;
((S)-1-{(S)-2-[5-(4′-{3-[(S)-1-(S)-2-Methoxycarbonylamino-3-methyl-butyryl)-pyrrolidine-2-carbonyl]-ureidomethyl}-biphenyl-4-yl)-1H-imidazol-2-yl]-pyrrolidine-1-carbonyl}-2-methyl-propyl)-carbamic acid methyl ester;
((S)-1-{(S)-2-[5-(4′-{3-[(S)-1-(R)-2-Methoxycarbonylamino-2-phenyl-acetyl)-pyrrolidine-2-carbonyl]-ureidomethyl}-biphenyl-4-yl)-1H-imidazol-2-yl]-pyrrolidine-1-carbonyl}-2-methyl-propyl)-carbamic acid methyl ester;
{(4S,7S)-4-[5-(4′-{3-[(S)-1-((R)-2-Methoxycarbonylamino-2-phenyl-acetyl)-pyrrolidine-2-carbonyl]-ureido}-biphenyl-4-yl)-1H-imidazol-2-yl]-6,10-dioxo-octahydro-pyridazino[1,2-a][1,2]diazepin-7-yl}-carbamic acid methyl ester;
(S)-2-{3-[2-(4-{2-[(S)-1-(S)-2-Methoxycarbonylamino-3-methyl-butyryl)-pyrrolidin-2-yl]-3H-imidazol-4-yl}-phenyl)-benzothiazol-6-yl]-ureidocarbonyl}-pyrrolidine-1-carboxylic acid tert-butyl ester;
[(S)-1-((S)-2-{5-[4-(6-{3-[(S)-1-((S)-2-Methoxycarbonylamino-3-methyl-butyryl)-pyrrolidine-2-carbonyl]-ureido}-benzothiazol-2-yl)-phenyl]-1H-imidazol-2-yl}-pyrrolidine-1-carbonyl)-2-methyl-propyl]-carbamic acid methyl ester;
[(S)-1-((S)-2-{5-[4-(5-{3-[(S)-1-((R)-2-Methoxycarbonylamino-2-phenyl-acetyl)-pyrrolidine-2-carbonyl]-ureido}-pyridin-2-yl)-phenyl]-1H-imidazol-2-yl}-pyrrolidine-1-carbonyl)-2-methyl-propyl]-carbamic acid methyl ester;
[(S)-1-((S)-2-{5-[4-(6-{3-[(S)-1-((R)-2-Dimethylamino-2-phenyl-acetyl)-pyrrolidine-2-carbonyl]-ureido}-benzothiazol-2-yl)-phenyl]-1H-imidazol-2-yl}-pyrrolidine-1-carbonyl)-2-methyl-propyl]-carbamic acid methyl ester; and
((S)-1-{(S)-2-[5-(3′-Fluoro-4′-{3-[(S)-1-((S)-2-methoxycarbonylamino-3-methyl-butyryl)-pyrrolidine-2-carbonyl]-ureido}-biphenyl-4-yl)-1H-imidazol-2-yl]-pyrrolidine-1-carbonyl}-2-methyl-propyl)-carbamic acid methyl ester.

12. A method for treating a Hepatitis C Virus (HCV) infection comprising administering to a patient in need thereof a therapeutically effective amount of a compound of claim 1.

13. The method of claim 12 further comprising administering an immune system modulator or an antiviral agent that inhibits replication of HCV, or a combination thereof.

14. The method of claim 13, wherein the immune system modulator is an interferon or chemically derivatized interferon.

15. The method of claim 13, wherein the antiviral agent is selected from the group consisting of a HCV protease inhibitor, a HCV polymerase inhibitor, a HCV helicase inhibitor, a HCV primase inhibitor, a HCV fusion inhibitor, and a combination thereof.

16. A method for inhibiting replication of HCV in a cell comprising administering a compound of claim 1.

17. A composition comprising a compound of claim 1 and a pharmaceutically acceptable excipient.
